(19) United States
(12) Patent Application Publication
(10) Pub. No.: US 2009/0036799 A1
(43) Pub. Date: Feb. 5, 2009

(54) Methods and Systems For The Diagnosis and Treatment of Medical Conditions in the Spine and Other Body Parts

(75) Inventors: Harvinder S. Sandhu, New York, NY (US); Jeffrey D. Schwardt, Palo Alto, CA (US); Richard W. Mott, Los Altos, CA (US)

Correspondence Address: HAYNES AND BOONE, LLP;IP Section, 2323 Victory Avenue, Suite 700, Dallas, TX, 75219, US

(73) Assignee: Medtronic Spinal and Biologics Business, Sunnyvale, CA (US)

(21) Appl. No.: 12/059,876

(22) Filed: Mar. 31, 2008

Related U.S. Application Data
(60) Provisional application No. 60/921,236, filed on Mar. 30, 2007.

(51) Int. Cl. A61B 5/103; A61B 19/00; A61B 5/00

(52) U.S. Cl. 600/587; 128/898; 600/301

(57) ABSTRACT

Disclosed are methods and systems for the diagnosis and treatment of medical conditions in the spine. In one variation, the method comprises measuring a first signal from a device positioned at or near a body part of a subject to determine a baseline signal, modifying a physiological condition of the body part, measuring a second signal from the device, and comparing the second signal to the first signal to determine whether a treatment is needed for the body part.

### STATEMENT OF RELATED APPLICATIONS

The present application claims priority under 35 USC 119(e) to U.S. provisional application No. 60/921,236, filed Mar. 30, 2007, entitled “Methods and Systems for the Diagnosis and Treatment of Medical Conditions in the Spine.” The disclosure of provisional patent application 60/921,236 is hereby incorporated by reference in its entirety herein.

### FIELD OF INVENTION

The present invention relates to methods and systems for the diagnosis and treatment of medical conditions in the spine.

### BACKGROUND

Back pain and other spinal ailments can take an enormous toll on the health of an individual, and can significantly reduce the productivity of people everywhere. Unfortunately, these spinal conditions are not only common, but may be difficult to accurately diagnose and treat.

A common back condition is discogenic back pain. Discogenic back pain originates in one or more intervertebral discs and can be particularly difficult to diagnose and treat. Many different factors may lead to discogenic pain, and it is often unclear whether treatment of a disc will impact the underlying causes of the pain that could remain after treatment. Furthermore, discogenic pain is difficult to pinpoint to one or more specific disks. The physical examination and complaints of the patient typically only provide general clues to the actual cause and source of the pain, and no currently available radiological methods exist to accurately assess which, if any, of a patient's discs are causing discogenic pain. Discogenic back pain is further described in U.S. Patent Publication 2005/0234425, application Ser. No. 10/825,961, published Oct. 20, 2005. The disclosure of U.S. Patent Publication 2005/0234425 is incorporated by reference in its entirety herein for all purposes.

Thus, one of the complications in treating these types of spinal aliments is that it is often difficult to identify the specific spinal level that is causing the pain. For example, various discography techniques have been developed to assist doctors in locating the source of spinal pain. However, typically these techniques rely on the patient's perception of pain. This may complicate the diagnosis procedure since the sensing of pain is subjective, such that different people perceive pain differently. In addition, in certain situations, the location or part of body where pain is perceived may not be the actual location where the ailment causing the pain resides, and therefore further complicates the diagnosis process.

Furthermore, individuals may have different pain thresholds and interpret the same objective amount of pain differently. Also, the subjective perception of pain may depend on psychological or environmental variables, such that the same individual may perceive pain differently depending upon the circumstances surrounding the occurrence of the pain. Pain may be subjectively assessed using a pain score such as the visual analog scale (VAS). However, for a scale from 1 to 10, the same amount of pain may be interpreted as a “9” by one person, but as a “4” for another.

Another common back condition that impacts many individuals is spinal stenosis. Spinal stenosis is a progressive narrowing of the spinal canal that causes compression of the spinal cord. As the spinal canal narrows, the spinal cord and nerve roots extending from the spinal cord and between adjacent vertebrae can be compressed and may become inflamed. Spinal stenosis can cause pain, weakness, numbness, burning sensations, tingling, and in particularly severe cases, may cause loss of bladder or bowel function, or paralysis. The legs, calves and buttocks are most commonly affected by spinal stenosis, however, the shoulders and arms may also be affected.

Mild cases of spinal stenosis may be treated with rest or restricted activity, non-steroidal anti-inflammatory drugs (e.g., aspirin), corticosteroid injections (epidural steroids), and/or physical therapy. Because spinal stenosis is a progressive disease, the source of pressure may have to be surgically corrected (decompressive laminectomy) if the subject develops increasing pain. The surgical procedure can remove bone, intervertebral discs (or portions thereof), and other tissues that impinge upon the spinal canal or put pressure on the spinal cord. Two adjacent vertebrae may also be fused during the surgical procedure to prevent an area of instability, improper alignment or slippage, such as that caused by spondylolisthesis. Surgical decompression can relieve pressure on the spinal cord or spinal nerve by widening the spinal canal to create more space. However, proper diagnosis and identification of the specific level and location that is causing pain is also important for the treatment of this condition.

The variations in pain perception from individual to individual can make it difficult for a health care provider relying on patient's subjective feedback to provide a diagnosis. In addition, it can be very difficult to objectify the actual pain perceived by the patient based on oral or written communication delivered by the patient. This can make it difficult for a health care provider to determine the correct treatment protocol for any given subject. Also, there may be times when a subject is less than completely forthcoming to the health care provider about the level of pain actually felt, as for example, due to a dependency on pain medication, or a desire to prolong a leave of absence from work.

Therefore, there is a need for an objective system for diagnosis of pain and other clinical indications that arises from the spine or other body parts. Furthermore, a feedback system that utilizes an objective parameter to provide a diagnosis of pain (e.g., locate the specific area or tissue causing the pain) or provide treatment and/or intervention of ailments in complicated body part such as the spine, can be desirable. In addition, in certain clinical conditions, the location with the ailment causing pain and the location where pain is perceived by the patient may not be the same. Therefore, an apparatus that is able to assist the physician to detect and interpret physiological response and correlated the location and/or pattern of the detected signal to specific location on the body requiring treatment (e.g., the specific level on the spinal column), could be useful for the treatment of the clinical condition.

### SUMMARY OF THE INVENTION

Embodiments of the present invention disclosed herein comprise methods and systems for the diagnosis and treatment of medical conditions in the spine and other body parts. The system may include computers and other microprocessor-based devices to assist the physician with the diagnosis and/or treatment process.

For example, in one embodiment, the present invention may comprise a method, the method comprising the steps of measuring a first signal from a device positioned at or near a body part of a subject to determine a baseline signal, modifying a physiological condition of the body part, measuring a second signal from the device, and comparing the second signal to the first signal to determine whether a treatment is needed for the body part.

In another embodiment, the present invention comprises a method, the method comprising the steps of positioning a device at or near a body part associated with pain, the device configured to modify a physiological condition of the body part. The method additionally comprises the steps of modifying the physiological condition of the body part, and determining a location of a body part causing the pain based at least in part on a physical response to the physiological modification.

In one embodiment, the method may comprise receiving a first signal associated with a physical response, where the first signal is received via a device positioned at or near a body part believed to be causing or associated with the generation of pain. The method may also comprise determining an objective level of pain based at least in part on the electrical signal. The method may further include actively inducing or suppressing pain by delivery a substance or electrical/mechanical excitation, while a subsequent physical response is monitored to determine whether such inductive or suppressive action modulates the output of the sensor. With such a feedback system, one may identify the specific location that is generating the pain.

For example, in another embodiment, the present invention may comprise an apparatus for objectively diagnosing and treating an ailment and/or pain in a subject. In one embodiment, the ailment causes the pain. The apparatus may comprise one or more sensors configured to monitor one or more levels of electrical activity at or near a body part believed to be causing, or associated with, the generation of pain, where each sensor is configured to generate a signal associated with a level of electrical activity at or near the body part. Also, the apparatus may comprise a processor in communication with the one or more sensors, such that the processor is configured to receive one or more signals from the one or more sensors. In certain embodiments, the processor may be configured to determine how the level of electrical activity at or near the body part corresponds to a physiological response which may be correlated with the location of the ailment and/or the pain. The apparatus may be further configured with a system to interfere or suppress the pain caused by a specific ailment. For example, a drug delivery system may be configured with to deliver anesthetic to various locations in the body part. The drug delivery system them selectively introduces anesthetic to the different locations as physiological response and/or pain perception is detected and/or measured. By identifying a specific location in the body part where injection of the anesthetic successfully suppresses the physiological response and/or pain, the physician may be able to locate the portion of the body part that has the ailment which causes pain.

In yet another embodiment, the present invention may comprise a computer-readable medium that comprises code for carrying out a method of the present invention for measuring pain and/or diagnosing the location causing the pain in a subject.

Other embodiments and further details on various aspects of the present invention are set forth in the following description, figures, and claims. It is to be understood that the invention is not limited in its application to the details set forth in the following description, figures, and claims, but is capable of other embodiments and of being practiced or carried out in various ways.

### BRIEF DESCRIPTION OF THE FIGURES

FIG. 1 is a block diagram illustrating a device for diagnosing and/or treating medical conditions in a body part in accordance with one embodiment of the present invention.

FIG. 2 is a block diagram illustrating an alternate device for diagnosing and/or treating medical conditions in a body part in accordance with another embodiment of the present invention.

FIG. 3 is a first illustration of a configuration for diagnosing and/or treating spinal pain in accordance with one embodiment of the present invention, where anesthetic delivery catheters are placed at multiple levels of the spine and electrical probes are positioned along the spine to detect electrical signals from muscles surrounding a plurality of intervertebral discs in the spine.

FIG. 4 is a flow diagram illustrating a first method for the diagnosis and treatment of medical conditions in a body part in accordance with one embodiment of the present invention.

FIG. 5 is a flow diagram illustrating a second method for the diagnosis and treatment of medical conditions in a body part in accordance with one embodiment of the present invention.

FIG. 6 is a flow diagram illustrating a third method for the diagnosis and treatment of medical conditions in a body part in accordance with another embodiment of the present invention.

FIG. 7 is a flow diagram illustrating a fourth method for the diagnosis and treatment of medical conditions in a body part in accordance with another embodiment of the present invention.

FIG. 8 is a second illustration of a configuration for diagnosing and/or treating spinal pain in accordance with one embodiment of the present invention, where a drug delivery device is placed at multiple levels of the spine and EEG probes are positioned along the head to detect signals.

FIG. 9 is a first illustration of a device for diagnosing and/or treating spinal pain in accordance with one embodiment of the present invention.

FIG. 10 is a second illustration of a device for diagnosing and/or treating spinal pain in accordance with one embodiment of the present invention.

FIG. 11 is a third illustration of a device for diagnosing and/or treating spinal pain in accordance with one embodiment of the present invention.

FIG. 12 is a fourth illustration of a device for diagnosing and/or treating spinal pain in accordance with one embodiment of the present invention.

### DETAILED DESCRIPTION

Unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

The term “treating” or “treat” refers to improving a symptom of a disease or disorder and may comprise curing the disorder, substantially preventing the onset of the disorder, or improving the subject's condition. The term “treatment” as used herein, refers to the full spectrum of treatments for a given disorder from which the subject is suffering, including alleviation of one symptom or most of the symptoms resulting from that disorder, a cure for the particular disorder, or prevention of the onset of the disorder. The term treatment may refer to the administration or application of remedies to a subject to relieve a medical condition or pain.

The term “therapeutic agent” is used to denote an agent that is intended to elicit a therapeutic response of an animal or human that is being sought. As used herein, an “effective amount” means the amount of an agent that is effective for producing a desired effect in a subject. The term “therapeutically effective amount” denotes that amount of a drug or pharmaceutical agent that will elicit a therapeutic response of an animal or human that is being sought. The actual dose which comprises an effective amount or a therapeutically effective amount may depend upon the route of administration, the size and health of the subject, the disorder being treated, and the like.

As used herein, a subject is an animal. For example, the subject may comprise a mammal. In one embodiment, the subject may be a human. A subject may be referred to as a patient. For example, a patient may be a subject who receives medical attention, care, or treatment. The operator or user of the products, methods, and systems of the present invention may be a physician, veterinarian, a health care professional, or another person or device.

As used herein, an internal body part may comprise a bone or bones, or part of a bone. The body part may comprise a portion of a spine, such as a vertebral body or an intervertebral disc. For example, due to various traumatic or pathologic conditions, an intervertebral disc can experience expansion or displacement leading to compression of the nerve and discomfort. The present invention is not, however, limited in application for treatment of pain, and may be used treat or diagnose other medical conditions (e.g., spasms, numbness, degenerated discs, etc.) in the spinal region.

Also, as used herein, an access member comprises a device for accessing a predetermined location in a subject. The inner lumen of the access member may provide a path to access a region or a body part that is located within the subject's body. The access member may be any type of device that can extend from the location of interest (e.g., the spine) to be accessible to a user of the access member. For example, the access member may be designed to extend from an internal body part in a subject to outside of the subject's body. The access member may be an elongated hollow member such as a hollow cylinder, a tube, a cannula, or a delivery catheter.

The terms “proximal” and “distal” refer to directions that are closer to, and away from, respectively, an operator (e.g., surgeon, physician, nurse, technician, etc.) who would insert a device of the present invention into the subject. Thus, for example, the end of an access member inserted inside the subject's body would be the distal end of the access member, while the end of the access member outside the subject's body would be the proximal end of the access member.

The term “processor” or “data analyzer” may refer to a device capable of executing computer-executable program instructions. Such processors may include one or more microprocessors, ASICs, and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices. Such processors include, or can be in communication with, media which stores instructions that when executed by the processor, cause the processor to perform the steps described herein. Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media can transmit or carry instructions to a computer, including a router, private or public network, or other transmission device or channel, both wired and wireless. The instructions can comprise code from any suitable computer-programming language, including, for example, C, C+, C++, Visual Basic®, Java™, Python™, and JavaScript®.

As used herein, the term “signal” refers to a discrete biological or electrical event that can be detected and in some cases, quantified. A signal may be sent from one cell to another, as in the case of transmission of electrical signals by nerve and/or muscle tissue. A signal may be transmitted from one place to another via electronic means, such as through wires. Alternatively, signals may be sent wirelessly.

As used herein, a “baseline signal” or a “control signal” may refer to a signal representative of the detected electrical level of a body part not undergoing pain, not being excited, or not being perturbed.

As used herein, the term “muscle signal” is a signal derived from a muscle cell or tissue.

As used herein, an “electrical signal” is a signal that comprises an electrical impulse (e.g., is derived from transmission of charged particles).

As used herein, a “treatment signal” is a signal that indicates that a therapeutic agent is to be administered to a body part being monitored for pain.

As used herein, the term “objective level of pain” refers to a level of pain uninfluenced by the subjective opinion of a subject describing the pain. In some variations, data collected for an objective level of pain may be quantifiable and be able to be compared with data collected at an earlier time from the same subject. The objective level of pain may also be quantifiable and be able to be compared across different subjects on the same scale.

As used herein, the term “subjective level of pain” refers to pain as perceived by the subject, and may be independent of any objective measurement.

The term “physiological state” may refer to the physical and/or biochemical state of a cell, tissue, organ, or other parts of a subject's body. For example, the physiological state of the spine and surrounding tissue may be changed through movement of the spine to thereby change the physical positioning of the spine, and/or through medical treatment such as treatment of the spine with a drug to thereby change the biochemistry of at least some of the cells that make up the spine.

As used herein, the term “sensor” may refer to any device, detector, transducer, and probes, such as an electromyography (EMG) electrode or an electroencephalography (EEG) electrode, which can be configured to detect a physiological state, such as the electrical activity, pressure, or some other state at or near a body part.

One of ordinary skill in the art having the benefit of this disclosure would appreciate that the present invention is not limited in its application to the details set forth in the description and figures but is capable of other embodiments and of being practiced or carried out in various ways.

Embodiments of the present invention provide methods, devices, systems, and computer-readable media for the diagnosis and treatment of pain and/or related medical conditions in the spine and other body parts. Certain embodiments of the present invention comprise the identification of a specific body part that requires medical treatment (e.g., locating a specific spinal level that has discogenic pain). For example, embodiments of the present invention may comprise methods, devices, systems and/or computer-readable media that measure electrical activity, pressure, or other conditions at or near a body part to sense pain relating to that body part. The methods, devices, systems and/or computer-readable media may further comprise components to objectively determine a subject's pain level so as to aid in the diagnosis and/or treatment of conditions related to a medical disorder. For example, the method may be utilized to identify a degenerated disc at a specific spinal level that needs to be treated (e.g. implant of an artificial disc, fusion, etc.) Other embodiments of the present invention may comprise methods, devices, systems and/or computer-readable media for the treatment of pain. The present invention may be embodied in a variety of ways.

In one embodiment, the present invention comprises a method, the method comprising the steps of measuring a first signal from a device positioned at or near a body part of a subject to determine a baseline signal, modifying a physiological condition of the body part, measuring a second signal from the device, and comparing the second signal to the first signal to determine whether a treatment is needed for the body part.

In one variation, a method of the present invention comprises the steps of positioning a device at or near a body part associated with a pain, the device configured to modify a physiological condition of the body part, modifying the physiological condition of the body part, and determining a location of a body part causing the pain based at least in part on a physical response to the physiological modification.

In another embodiment, the present invention may comprise a method to objectively diagnose and/or measure pain in a body part. The method may comprise positioning a first sensor at or near a portion of an internal body part believed to be causing or associated with the generation of pain; receiving a first signal via the at least one first sensor, the first signal associated with a first level of electrical activity at or near the portion of the body part believed to be causing or associated with the generation of pain, and determining an objective level of pain based at least in part on the electrical signal.

In some cases, a baseline (e.g., control) reading corresponding to a body part not undergoing pain is observed. For example, a baseline reading may be generated by a sensor placed on a part of a subject's body which does not generate any pain. Alternatively, a baseline reading may be captured by the same sensor later used to detect pain. For example, a baseline reading may be detected by a sensor while a subject is at rest and not experiencing pain. A baseline reading may be recorded for later use.

In one variation, a baseline reading or reference is established while the patient is asked to assume a specific position that generates pain. Readings or signals captured after the patient has undergone a specific treatment, intervention, or modification of a physiological condition (e.g. introduction of analgesic) are then compared to the baseline reading to determine if the treatment or intervention has an effect on the patient.

It may be important to compare the physical response of a body part body part being examined (i.e., a body part believed to be causing or associated with the generation of pain) to a body part that is known to be disease free and/or pain free. The second sensor may thereby comprise a negative control or normalized level of electrical activity. In that way, unusual electrical activity that is occurring at the body part of interest may be detected. For example, in one embodiment, the method may comprise positioning a sensor at or near a portion of a body part believed to be free of pain, and receiving a control signal, the control signal associated with a baseline level of electrical activity at or near the portion of the body part believed to be free of pain; and comparing the first signal and the control signal to provide a level of electrical activity caused by, or associated with, the generation of pain.

In some cases, more than one sensor may be used to evaluate the body part of interest. Thus, in certain embodiments, the method may comprise using a plurality of first sensors positioned at or near a portion of an internal body part believed to be causing or associated with the generation of pain, and comparing the signals received from the plurality of sensors to provide a level of electrical activity caused by or associated with the generation of pain. In an embodiment, the signals received from the plurality of sensors positioned at or near a portion of an internal body part believed to be causing or associated with the generation of pain are compared to the signal from a control sensor, where the control sensor is positioned at or near the portion of the body part believed to be free of pain.

In certain embodiments, the method may comprise analyzing the subject's subjective valuation of pain in conjunction with the measurement of electrical activity at the body part believed to be causing or associated with the generation of pain. The method may further comprise correlating the level of electrical activity at the body part (in some cases as compared to a control reading one of the first sensors or from a second sensor) to the subject's subjective perception of pain.

In one embodiment, the level of electrical activity and/or the objective level of pain may be correlated to a change in the physiological state of the body part. Thus, the method may comprise receiving a plurality of signals from the first sensor, wherein the signals are received at different times. The method may further comprise determining how the change in the signal or signals received from the first sensor at a first time point as compared to the signal or signals received at a second time point corresponds to a change in the physiological state of the body part at the first time point as compared to the second time point.

The measurement of electrical activity may be correlated to a variety of changes that can occur in the physiological state of the body part of interest. The change in the physiological state of the body part may comprise the state of the body part before and after the subject has undergone repositioning of the body part. Or, the change in the physiological state of the body part may comprise the state of the body part before and after the subject has undergone a medical procedure to treat the body part. For example, where the body part is a spine, or a portion of a spine, the change in physiological state may be the spine before and after surgery. In another embodiment, the change in the physiological state of the body part may comprise the state of the body part before and after the subject has been treated with a drug. For example, the change in the state of the body part may comprise the body part before and after treatment with a pain suppressor (e.g., delivery of an analgesic or anesthetic into or around the body part). In yet another embodiment, the change in the physiological state of the body part comprises the state of the body part before and after an implant has been placed into or adjacent to the body part.

A variety of body parts may be targeted with a method, apparatus, or system of the present invention. In certain embodiments, the body part is at least a portion of the spine of the subject. In one embodiment, the body part is an intervertebral disc. Or, the body part may comprise a vertebral body. For example, the physiological state of the body part may comprise a change in the physical positioning of at least a portion of the spine (e.g., an intervertebral disc or an intervertebral disc in combination with adjacent vertebral bodies). In one embodiment, the portion of the spine that is repositioned may comprise the portion of the spine that is monitored for electrical activity. Or, the change in the state of the spine may comprise the state of the spine after the subject has been treated with a drug. For example, the change in the spine may comprise the physiological state of the spine, or a portion of the spine, after treatment with an anesthetic or analgesic. Or, a combination of changes in the physiological state of the body part may be assessed (e.g., treatment of the spine with a pain killer in conjunction with a change in position of the spine or a portion of the spine). In another variation, the change in the state of the spine may comprise the state of the spine after a device has been implanted in or adjacent to the spine.

A variety of methods for monitoring electrical activity may be used in the methods of the present invention. In certain embodiments, electromyography (EMG) is used to monitor the electrical activity of muscles at or near the body part of interest. As is known in the art, EMG may be used to evaluate physiological conditions or disorders including, but not limited to, neuromuscular disorders; motor problems, such as involuntary muscle twitching; nerve compression or injury; nerve root injury; and muscle degeneration. Both the amplitude, frequency, pattern, and/or the shape of the resultant EMG signal may be used to detect changes in physiological conditions and/or abnormalities in a muscle.

EMG may measure muscle response or electrical activity in response to a nerve's stimulation of a muscle. Thus, EMG may be used to measure the electrical activity of muscle during rest, slight contraction, and forceful contraction. During an EMG test, one or more small needles, conduction pads, or electrodes, may be inserted through a subject's skin into the muscle or attached to the surface of the skin. The electrical activity picked up by the electrodes may then be displayed on an oscilloscope or other monitor that displays electrical activity. An audio-amplifier may also be used to provide an audible signal that relates to the electrical activity. For example, where the body part being monitored is the spine, an EMG may be used to measure the electrical activity in the paraspinal muscles. In an embodiment, EMG measurements may comprise signals in the millivolt range. For example, in alternate embodiments, once the EMG signals are amplified and/or filtered as described in detail herein, signals between about 0.5 Hertz and 2 Hertz may include the bulk of pain intensity information. Thus, ranges from about 0.05 to about 10 Hertz, or from about 0.1 to about 6 Hertz, or from about 0.5 to about 2.5 Hertz may be used.

In another embodiment, nerve conduction velocity (NCV) may be used to in the diagnosis and/or measurement of a physiological condition or parameter. In one variation, an EMG in conjunction with NCV may be used. A NCV test can assess how well a specific nerve conducts impulses by evaluating the speed of an impulse as it travels along a nerve. The test can help determine if there is nerve damage, the extent of the damage, and/or if nerves have been destroyed. For example, an NCV may be used to determine how well the nerve roots leaving the spine are working. For an NCV, patch-like electrodes, similar to those used during an electrocardiogram, may be affixed on the skin at various nerve locations. A probe held against the skin may then be activated to emit a very low electrical impulse to stimulate the nerve. The electrodes may then measure the speed of the impulse as it travels from one point to a second point, and nerve activity is recorded and displayed on a CRT screen. For example, NCV may be utilized to detect a problem with the nerve, while EMG is utilized concurrently or sequentially to detect diseases stemming from problems with the muscle itself, as well as other problems that result from influences on the muscle from other systems, such as nerves. In another variation, NCV detection is used in combination with EMG monitoring. Electrical probes or pin-like insertable electrodes may also be used in place of patch-like electrodes for NCV monitoring.

In other embodiments, electroencephalography (EEG) may be used to monitor electrical activity in the brain. The EEG signal may then, in certain embodiments, be correlated to pain perception caused by activities around the spine. In an embodiment, the EEG signal may be in the tens of microvolt range (e.g., about 10-99 microvolts).

Other methods of determining nerve activity may also be used. For example, in certain embodiments, an evoked potential (EP) comprising an electrical potential may be recorded following a stimulus. In certain embodiments, a somatosensor evoked potential (SSEP) may be determined. For example, a SSEP may be used to detect a pinched nerve, since if nerves are pinched, the signal generally will travel slower than expected. For EP and/or SSEP, the evoked potentials may be in the sub-microvolt to several microvolts (e.g., 0.1 to 50 microvolts).

The level of electrical activity and/or the objective level of pain may be correlated to a particular medical treatment or therapy. In certain embodiments, a treatment may be administered based on the level of electrical activity measured at or near the body part. For example, a treatment may be administered if the level of electrical activity resulting from pain exceeds a certain objective threshold. The objective threshold may indicate that a subject would benefit from the administration of a particular treatment.

The treatment may be administered by a health care professional based on an evaluation of the measured electrical activity at the body part of interest. Or, as discussed in more detail herein, the treatment may be automatically administered. For example, a processor may automatically generate a signal (i.e., a “treatment signal”) activating a drug delivery system if an electrical level indicated by a muscle signal exceeds a predetermined threshold. The treatment signal may then be transmitted to a treatment device. In an embodiment, the treatment device comprises at least one catheter emplaced at or near the body part and configured to distribute the therapeutic agent to the body part.

In certain embodiments, the methods of the present invention may provide a feedback system for the automated diagnosis and treatment of pain. For example, the invention may comprise a method for the automated treatment of pain. Thus, in one embodiment, the method may comprise measuring one or more electrical activities at a body part, and then automatically dispensing a therapeutic agent if the level of pain exceeds a certain threshold. In certain embodiments, after a first measurement is performed, followed by the administration of a therapeutic agent, the body part may be physiologically altered, and then the electrical activity at or around the body part is again measured. If, the level of pain still exceeds a certain threshold, a therapeutic agent may again be dispensed. In one embodiment, the body part is a spine or a portion of a spine (e.g., an intervertebral disc). For example, an EEG and/or EMG may be measured before and after the condition at a specific disc has been altered (e.g., through injection of an analgesic, a spinal fusion, or other spinal procedures) to determine if the alteration has an effect. One or ordinary skill in the art having the benefit of this disclosure would appreciate that other physiological parameters (e.g. pressure load, blood flow, etc.) may also be monitored to determine the change in physiological condition of the body part.

In other embodiments, the present invention may comprise an apparatus or system for objectively diagnosing and/or treating pain in a body part. The apparatus may comprise one or more sensors configured to monitor one or more levels of electrical activity at or near a body part believed to be causing, or associated with, the generation of pain, where each sensor is configured to generate a signal associated with a level of electrical activity at or near the body part; and a processor in communication with the one or more sensors, where the processor is configured to receive one or more signals from the one or more sensors. In certain embodiments, the processor may be further configured to determine how the level of electrical activity at or near the body part corresponds to an objective level of pain.

In one embodiment, the present invention may comprise a device configured to detect or modify a physiological condition of a body part, the body part believed to be causing, or associated with, the generation of pain; and a processor in communication with the device, the processor configured to determine a location of a body part actually causing the pain based at least in part on a physical response to a modification of the physiological condition of the body part.

Thus, the apparatus or system may comprise at least one component to receive the electrical activity at or near the body part. In an embodiment, the component to receive the electrical activity is a detector. The apparatus or system may also comprise at least one component for emplacing the detector at or near the body part in the subject.

The detector may, in certain embodiments, comprise a sensor configured to monitor a level of electrical activity at or near a body part. In one embodiment, the sensor may comprise an electrode. For example, electromyography (EMG) electrodes or conductive pads may be used. Alternatively, electroencephalography (EEG) electrodes may be used. Or, other electrodes (e.g., NCV electrodes) known in the art of monitoring physiological (e.g., nerve and muscle) electrical activity may be used.

In some cases, more than one detector may be emplaced at or near the body part. Thus, in one embodiment, the apparatus may comprise one or more sensors configured to monitor one or more levels of electrical activity at or near one or more body parts.

The apparatus or system may also comprise components for correlating how the level of electrical activity corresponds to an objective level of pain or a physiological parameter being monitored. Thus, the sensor and/or the system may comprise components to transform the signal as it is received into a form that can be received and/or analyzed by the processor.

For example, in one embodiment, the apparatus may comprise an amplifier or amplifiers configured to amplify a signal received by the sensor. Also, the apparatus or system may comprise a filter (or filters) to remove background noise. In yet other embodiments, the apparatus or system may comprise a converter to transform analog signals to digital signals.

The apparatus or system may also comprise a processor. For example, the apparatus or system may comprise a processor in communication with the one or more sensors. The processor may comprise an electronic process (or other computation device) configured to evaluate the signals received by the sensors. In one embodiment, the processor may be configured to receive one or more signals from the one or more sensors. Also, the processor may be configured to correlate the level of electrical activity at or near a body part to an objective level of pain.

The apparatus or system may further comprise an input terminal in communication with the processor. The input terminal may be configured to provide at least one parameter to the processor. For example, the input terminal may be used to transmit instructions and/or analytical parameters to the processor.

In one embodiment, the apparatus or system may comprise a monitor. The monitor may provide a means for a user to evaluate the level of the signal received and/or transmitted by the sensor. For example, in one embodiment, the apparatus may comprise a monitor in communication with the processor, where the monitor is configured to show a visual representation of the signal or signals detected near the body part of interest. Thus, where the sensor is an EMG sensor, an electromyography (EMG) monitor may be used. Alternatively, where the sensor is an EEG sensor, an electroencephalography (EEG) monitor may be used. Or, other monitors known in the art of monitoring physiological (e.g., nerve and muscle) electrical activity may be used. In an embodiment, the monitor may be in communication with at least one sensor and the processor, such that the monitor is configured to receive an electrical signal from the sensor and provide a signal to the processor.

As described herein, the apparatus and/or system may comprise a component for treatment of the subject. The treatment may be based, at least in part, on the objective measurement and/or diagnosis of pain at the body part being monitored. In an embodiment, the apparatus may comprise a component for measuring how the level of electrical activity corresponds to a change in the state of the body part. Thus, certain embodiments of the apparatus or system may comprise a therapeutic delivery device configured to deliver a therapeutic agent to the body part. In one embodiment, the therapeutic delivery device may be in communication with the processor. In one embodiment, the therapeutic delivery device may comprise a programmable dispenser.

A variety of devices may be used to dispense a therapeutic agent to the body part. For example, the delivery device may deliver an electrical signal. Or, the delivery device may deliver a drug or therapeutic agent. In an embodiment, the device for delivering a therapeutic agent to the body part may comprise an access member. For example, the device for delivering a therapeutic agent to the body part may comprise a catheter, or a plurality of catheters.

The treatment may be administered by a physician. Or, the treatment may be automatically administered based on the level of the electrical activity measured at or near the body part. For example, in certain embodiments, the apparatus or system may comprise a processor to control the dispensing of a therapeutic agent to a body part. For example, a processor may automatically generate a signal (i.e., a “treatment signal”) activating a drug delivery system if an electrical level indicated by a muscle signal exceeds a predetermined threshold.

In yet other embodiments, the present invention may comprise computer software to automate and control systems for diagnosing and treating pain in a body part. Thus, certain embodiments of the present invention may comprise computer readable medium on which is encoded computer-executable program code for determining an objective level of pain. The program code may comprise code for receiving a first signal via a sensor, where the first signal is associated with a first level of electrical activity at or near the portion of the body part believed to be causing or associated with the generation of pain; and program code for determining an objective level of pain based at least in part on the electrical signal.

For example, in one embodiment, the present invention comprises a computer readable medium on which is encoded computer-executable program code to diagnose a location of pain in a subject, the program code comprising: program code for receiving a first signal via a device, the first signal associated with a first level of a physical response at or near the portion of a body part believed to be causing, or associated with, the generation of pain; and program code for determining a location of a body part actually causing the generation of pain based at least in part on the physical response.

In some cases, it may be important to compare the electrical activity near the body part being examined (i.e., a body part believed to be causing or associated with the generation of pain) to the electrical activity at a body part that is known to be disease and/or pain free. (e.g., a “control” or normalized level of electrical activity). In that way, unusual or abnormal electrical activity that is occurring at the body part of interest may be detected. Thus, in one embodiment, the computer-readable medium may further comprise program code for receiving a control signal via a control sensor, the control signal associated with a level of electrical activity at or near the portion of a body part believed to be free of pain; and comparing the first signal and the control signal to provide a level of electrical activity caused by, or associated with, the generation of pain.

In one variation, a first signal collected at the first location having a healthy first body part is used as the baseline (i.e. reference). A second signal collected at a second location having a second body part with a suspected ailment is collected and compared with the first signal to determine if the second body part is healthy or requires treatment. In an alternative embodiment, a first set of signals are measured by a first body part and a second body part. Next, the physiological conditions of the first body part and the second body part are altered (e.g. introduction of an anesthetic, introduction of a therapeutic agent, implantation of a device, modification of pressure, etc.). After the alteration, a second set of signals are collected from the first body part and the second body part. The signals from the first body part and the second body part are then compared to each other to determine if the second body part is healthy or requires treatment. The first set of signals may also be utilized during the comparison to determine whether the second body part requires treatment. In one example the body parts are intervertebral discs.

The computer-readable medium may further comprise code to compare the signals received from a plurality of sensors, where the signals are associated with electrical activity at or near the portion of a body part believed to be causing or associated with the generation of pain to provide a level of electrical activity caused by or associated with the generation of pain. In an embodiment, the software comprises code to compare the signals received from the plurality of sensors positioned at or near a portion of an internal body part believed to be causing or associated with the generation of pain to the signal from a control sensor, where the control sensor is positioned at or near the portion of the body part believed to be free of pain.

In an embodiment, the first sensor and the control sensor are the same sensor. For example, the control sensor may provide a reading at the body part before the body part in manipulated to induce pain. In other embodiments, the control reading may be taken from a sensor than the first sensor positioned at the body part believed to be causing or associated with the generation of pain.

In an embodiment, the computer code may comprise instructions for transforming the signal as received from the sensor, or a downstream amplifier, filter and/or monitor, to a signal that can be quantified and correlated to an objective measure of pain. For example, in an embodiment, the signal may be digitized. Additionally or alternatively, the signal may be compared to a control reading (e.g., the signal from a second sensor) for removal of noise. In an embodiment, the signal may also be parsed into segments representing discrete electrical impulses or measurements. Additionally or alternatively, the signal may be normalized, undergo subtraction of the mean and/or other analyses as required. An example of signal processing scheme is disclosed in U.S. Pat. No. 6,826,426, which is incorporated herein by reference in its entirety for all purposes.

The level of electrical activity and/or the objective level of pain and/or parameter of a physiological condition may be correlated to a change in the physiological state of the body part.

Thus, the computer and/or the computer computer-readable medium may further comprise program code for receiving a plurality of signals from the first sensor, wherein the signals are received at different times. The code may further comprise determining how the change in the signal or signals received from the first sensor at a first time point as compared to the signal or signals received at a second time point corresponds to a change in the physiological state of the body part at the first time point as compared to the second time point. The change in the physiological state of the body part may be a change in the position of the body part, or the state of the body part after treatment with a drug or after a medical procedure (e.g. implantation of a device, or removal or alteration of a tissue, etc.).

The software may also be used to correlate the measured electrical signal to the subject's subjective evaluation of pain. Thus, in an embodiment, the computer code may comprise code for correlating the level of electrical activity detected at the body part believed to be causing, or associated with, the generation of pain, to a subject's subjective perception of pain.

The computer software of the present invention may be used to provide a method of treatment or as part of an apparatus or system for treatment of pain at or near a body part. For example, the computer program may comprise program code for determining whether medical intervention is needed based at least in part on the objective level of pain. The computer program may be designed to facilitate the automated treatment of the subject. In an embodiment, the computer program may comprise code for generating a treatment signal, where the treatment signal is configured to cause a distribution of a therapeutic agent to the body part. Alternatively, the treatment signal may result in transmission of an electric current or application of an electromagnetic wave to the tissue in the body part, or activation of a transducer which leads to motion or displacement of tissue in the body part.

For example, the computer program may comprise software to interpret data recorded by an apparatus or system of the present invention. In one embodiment, the present invention may comprise a computer program to control the automatic dispensing of a therapeutic agent to a body part. The program may be designed such that a therapeutic agent is infused into the body part if the electrical activity exceeds a predetermined threshold, wherein the predetermined threshold indicates that the subject would benefit from infusion of the therapeutic agent into the body part.

In certain embodiments, delivery of the therapeutic agent may require manual intervention. Or, there may be a maximum threshold provided, such that the therapeutic agent cannot be administered continuously for long periods of time, or at high doses, without the authorization of trained medical personnel. For example, where the therapeutic agent is a pain medication or anesthetic, there may be a need for a treatment procedure to be authorized by a physician and/or an override function to prevent unintentional over-dosage.

A method, apparatus, system and/or computer software of the present invention may be used for the diagnosis and/or treatment of pain, where the pain relates to, or is caused by, a variety of body parts. For example, as discussed herein, it can be difficult to diagnose the source of spinal pain. Thus, certain embodiments of the methods, apparatuses, systems and/or computer software of the present invention may be implemented to diagnose the source of pain in a spine, or a portion of a spine, such as an intervertebral disc. In one embodiment, the detector emplaced at or near the body part may comprise a device for monitoring one or more physiological parameters (e.g., contraction of the muscles, nerve excitation, brain wave, and the like) relating to the condition of an intervertebral disc before and after a condition where an intervertebral disc has been modified (e.g., injection of an analgesic, etc.).

For example, the physiological parameters of muscles surrounding the intervertebral disc can be monitored by Electromyography (EMG), which detects electrical signals generated by the muscles. In an embodiment, the electromyogram may provide data to a physician regarding the intensity of contractions and other parameters of the muscles surrounding the intervertebral disc. The subject (e.g., a patient) may be asked to assume various poses to change the position of their spine, and the EMG signal recorded as the subject assumes various poses. An analgesic or an anesthetic may then be injected into the intervertebral disc. After the injection of the analgesic, the subject may be asked to repeat the various poses to change the position of their spine. By monitoring the electromyogram, the physician can then determine if the analgesic changes the pattern of the signals emitted by the muscles surrounding the intervertebral disc. If the EMG is suppressed, this may suggest that the particular spinal level being monitored needs to be treated.

For example, local pain generated in, or around, a specific intervertebral disc may intensify the contraction and/or other physiological parameters of the muscles surrounding the intervertebral disc. As a result, electrical signals generated by the muscles can increase. Therefore, a subject assuming a position which generates pain in a specific disc, may cause an increase in the electrical signal generated by the surrounding muscle, resulting in a corresponding increase in the EMG signal amplitude and/or frequency. When the subject assumes the same position after an analgesic has been delivered into the disc, the electrical signal detected through the EMG may be suppressed, since the nerves in and/or surrounding the disc are suppressed. If the analgesic is injected into a disc which is not the source of the pain, the pain may persist and the EMG signal (and the pain) would not be suppressed. This approach can provide an objective and qualitative process to measure back-pain and identify the intervertebral disc that is causing pain.

In one variation, an catheter with an deployable anchor (e.g., a catheter having a balloon as an inflatable anchor, etc.) may be inserted into the intervertebral disc with the distal end of the catheter anchored in the disc prior to the monitoring of the EMG or other physiological parameter in the patient's body. Thereafter, the patient is instructed to assume one or more positions that generate pain as the baseline (i.e., reference) signal is measured. A substance, such as an anesthetic or an analgesic, is then injected into the intervertebral disc through the anchored catheter. After the substance has settled in the intervertebral disc, the patient is instructed to assume the one or more positions that had generated pain previously. As second signal is measure and compared with the baseline signal to determine if treatment is needed for the intervertebral disc.

Once the disc or portion of the spine that is causing pain has been identified, a therapeutic agent (e.g., an analgesic, anti-inflammatory, or other therapeutic agents used in the treatment of spinal conditions) may be injected into a spinal disc through a catheter anchored in the spinal disc if needed. In one embodiment, a catheter with a deployable anchor may be used to secure a distal portion of the catheter in the spinal disc. For example, a balloon catheter may be inserted into the spinal disc and the balloon inflated to secure the catheter in the spinal disc. Devices for injection of a therapeutic agent into a spinal disc are described in U.S. Patent Publication 2005/0234425 and are also commercially available (e.g., Kyphon, Inc.). Also, the methods, devices and/or systems of the present invention may be used in conjunction with other therapeutic methods (and combination therapies), devices and/or systems that may be used for treatment of the spine and/or other body parts. Therapeutic agents may include analgesics and anesthetics known in the art. For example, analgesics and anesthetics may include those described in Patent Publication US 2005/0234425, application Ser. No. 10/825,961, published Oct. 20, 2005, incorporated by reference herein in its entirety for all purposes.

In certain embodiments, the subject's subjective valuation of pain relating to a body part may be analyzed in conjunction with the objective measurement of electrical activity at or near the body part. For example, a subject's subjective valuation of the pain may be incorporated with the EMG data to determine which intervertebral disc is causing pain. Thus, the subject may be instructed to perform a task or assume a position that would induce pain in the spinal region. When the subject indicates a sensation of pain, the subjective valuation of pain may be correlated with the electrical signal recorded by the EMG system.

In one variation, the subject may be asked to rate the sensation of pain using a predefined scale (e.g., the subject may be asked to enter a number between 1 to 10 on a keypad). The pain detection information as indicated by the subject can be recorded along with the EMG signal. An analgesic may then be injected into the specific spinal disc to be evaluated. Once the analgesic has been given time to permeate the tissue, the subject may be asked to repeat the back-pain generating position, and rate the level of pain, if any. The subjective data provided by subject may be used in combination with the objective EMG data to diagnose the location of the disc which is causing spinal pain.

In other embodiments, the methods, apparatuses and systems of the present invention may comprise the use of a plurality of electrical detectors to measure electrical activity at or near a body part. For example, in one embodiment, an array of electrical conduction pads may be placed on the subject's back near the spine to measure EMG signals at various locations on the spine. In this way, a plurality of objective measurements of electrical activity may be evaluated for a single change in the physiological state of the spine.

The electrical monitoring device may be varied depending upon the diagnosis and/or therapy required. In one embodiment, the electrical monitoring is from muscle tissue such that an EMG is used. An EMG may be used to detect abnormal electrical activity in a muscle, where the abnormal electrical activity due to abnormality of the muscles or nerves that are near or innervate the muscles.

In an alternative approach, Electroencephalography (EEG) data may be collected to serve as an objective source for pain measurement. In one embodiment, the pain may be emanating from the subject's spine. Or headache or other body pain may be monitored. In the case of back pain, where the body part being monitored may be an intervertebral disc, the EEG data may be continuously recorded, and the subject asked to assume one or more back-pain generating positions. Once the subject has completed the first set of movements, an analgesic may be injected into the intervertebral disc suspected of causing pain. Thereafter, the subject may be asked to repeat the movements which caused pain. The EEG, before and after the injection of the analgesic, may be compared to determine if the targeted disc is the one that is causing pain. For example, parameters (e.g., amplitude, power, frequency, etc.) of an EEG signal from before and after the injection of the analgesic may be compare to determine if the injection result in detectable changes in the EEG signal. Suppression of the EEG signal after the injection of the analgesic may suggest that the treated disc is the source of back-pain. The steps may be repeated on one or more other intervertebral discs along the spine.

In other embodiments, the EEG measurements are combined with EMG measurements to diagnose the source of pain. Or, the EEG measurement may be combined with subject's subjective feedback. In yet another variation, the EEG data is combined with EMG and subject feedback for diagnosis of spinal pain. One of ordinary skill in the art having the benefit of this disclosure would appreciate that other electrical activities that can be associated with pain or a physical condition of the spine may also be implemented in the objective feedback system for pain diagnoses.

In some cases, a therapeutic agent may be infused into the body part if the electrical activity exceeds a predetermined threshold, wherein the predetermined threshold indicates that the subject would benefit from infusion of the therapeutic agent into the body part. In certain embodiments, a plurality of catheters may be emplaced at or near the body part and used in conjunction with a plurality of electrical monitoring devices. For example, an array of detectors may be implemented in combination with one or more catheters that are inserted into one or more intervertebral discs. The catheters may then be used to deliver substances into the discs. Traditional discography techniques and/or functional anesthetic discography techniques may be implemented in combination with methods described herein to diagnose the source of discogenic pain.

In other embodiments, a method, apparatus, and/or system of the present invention may be implemented to evaluate the effectiveness of a medical intervention. In certain embodiments, an electrical recording of electrical activity at or near a portion of the spine may be utilized to assess the results of a medical procedure performed on the spine. For example, an EEG and/or an EMG may be recorded prior to a spinal surgery while the subject is instructed to perform a series of predefined motions. After the surgery, such as a spinal fusion, the subject may be asked to repeat the same series of predefined motions as the EEG and/or EMG is being recorded. The data collected pre-operation and post-operation may then be utilized as benchmarks to evaluate the result of the surgery. One of ordinary skill in the art having the benefit of this disclosure would appreciate that the various detection methods and systems disclosed herein can be implemented to measure and/or detect various physiological conditions of the spine, and can also be implemented to determine the effectiveness of various treatments and medical interventions that are performed on the spine.

In certain embodiments, the detection system (e.g., EEG, EMG, etc.) may be coupled to a drug delivery system (e.g., drug pump, drug infusion device, etc.). In one embodiment, the drug delivery system may be automated. When a signal is received by the detection system indicating that pain is being experienced by the subject, the drug pump may be activated to deliver a medication.

The drug pump and the detection system may be implanted in the subject's body. Alternatively, the drug pump may be positioned external to the subject's body. For example, in one embodiment, one or more electrical leads may be positioned on the subject's body to detect the EMG around the subject's spine. For example, a detection system may be connected to the electrical leads to receive and analyze the EMG signal. A drug pump may be coupled to the detection system such that the drug pump can be modulated by the detection system. The distal end of a balloon catheter may be implanted in a spinal disc or intervertebral space in the subject's spine. The balloon at the distal portion of the catheter may be inflated to prevent accidental removal. The detection system may be configured to detect particular patterns of EMG signals that are correlated with pain in the spinal area. When an EMG signal pattern suggests that the subject is experiencing pain, the drug pump may be activated to deliver medication to suppress the pain in the spine. Thus, the proximal end of the catheter may be coupled to the drug pump, such that when an EMG signal is received by the detection device, an electrical signal is sent from the detection device to the drug pump to instruct the drug pump to infuse analgesic through the catheter into the spinal disc. Also, the system may also be configured to increase the amount of medication being infused into the spine when the intensity of the detect EMG signal is increasing. Alternatively, the system may be configured to continuously deliver a based-line level of analgesic. The system may then increase and/or decrease the level of analgesic being infused depending on the detected EMG signal. Additionally or alternatively, the system may comprise an override and/or warning if it appears that regulation of delivery of the therapeutic agent is required. One of ordinary skill in the art having the benefit of this disclosure would appreciate that the feedback drug delivery system disclosed above may also be configured to utilize EEG and/or other biological signals to control drug delivery for pain management and/or other medical treatments.

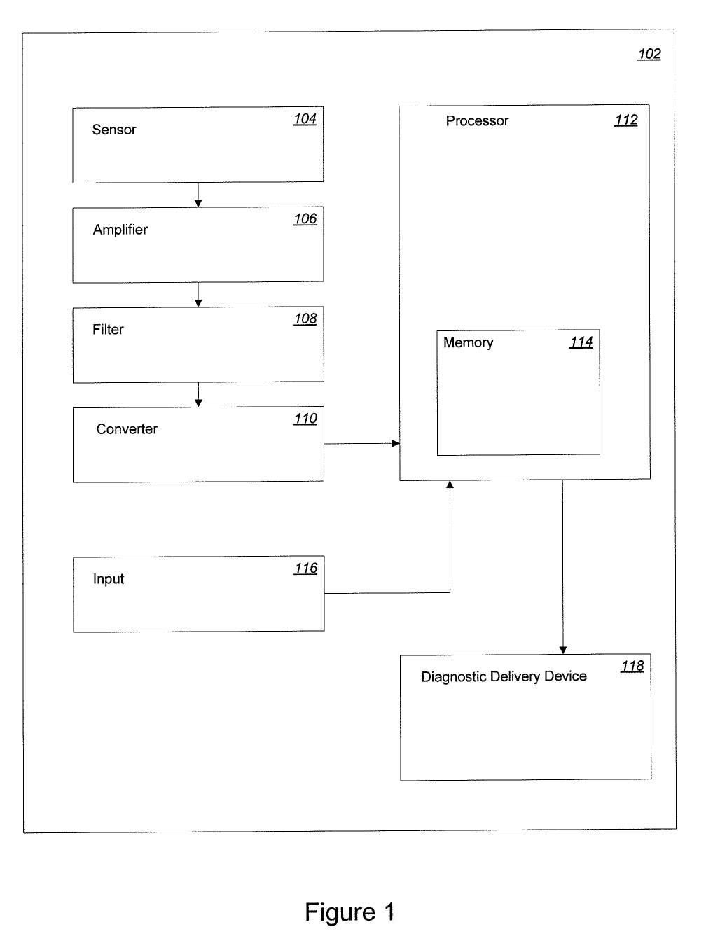

FIG. 1 is a block diagram illustrating a device for diagnosing and treating medical conditions in a body part in accordance with one embodiment of the present invention. In the embodiment shown, the device 102 comprises a detector (e.g., sensor) 104. In other embodiments, the device may comprise two, three, or more sensors. The sensor, or detector, may be operable to measure one or more physiological parameters (e.g., contraction of the muscles, nerve excitation, brain wave, etc.) relating to the body part being monitored. As a specific example, the sensor may be able to measure the electrical activity at or near an intervertebral disc.

In one embodiment, the sensor 104 may comprise an electrode. For example, electromyography (EMG) electrodes or conductive pads may be used. Alternatively, electroencephalography (EEG) electrodes may be used. Other electrodes known in the art of monitoring physiological (e.g., nerve and muscle) electrical activity may be used.

The sensors may transform the electrical signal to a signal that can be recognized by the apparatus. Thus, in an embodiment, the sensors (detectors) transform the electrical signal detected from the muscles or nerves near the body part, to a type of electrical signal that can be recognized by other components of the device. Thus, in certain embodiments, the signal received by the electrode is transmitted to an amplifier 106. In some embodiments the device may comprise a plurality of amplifiers 106.

The amplifier 106 may be part of the sensor 104, or it may be a separate component. In certain embodiments, the amplifier 106 may amplify the signal generated from the electrode 104 to a signal ranging from about 0 to 5 volts. Also, in some embodiments, the sensors 104 may comprise preamplifiers. Such preamplifiers may be configured to perform initial signal amplification without amplifying subsequently acquired noise contributions.

The device may further comprise a filter 108 or a plurality of filters 108 to remove electrophysiological artifacts, radio frequency transmission, and other electromagnetic noise. A filter 108 may be in communication with an amplifier 106. In some embodiments, a plurality of filters 108 are in communication with a plurality of amplifiers 106.

The system may also comprise a converter 110 which may be used to digitize the analog signals to digital signals. In one embodiment, the sampling at the converter should be at least 10 Hz, but may be increased to about 250 Hz to improve sensitivity (see e.g., U.S. Pat. No. 6,826,426 incorporated by reference herein in its entirety). In another embodiment, the sampling may be at a higher frequency. The converter 110 may be in communication with one or more filters 108.

Referring still to FIG. 1, the device 102 may also comprise a processor 112. The processor 112 may be in communication with the sensor 104 to receive raw electrical signals, or may be downstream of the amplifier 106, filter 108, and/or converter 110 to receive transformed electrical signals based at least in part on the signals are received by the sensors 104. In one embodiment, the processor 112 is in communication with the converter 110. In another embodiment, the processor 112 may be in communication with a plurality of converters 110, or a plurality of sensors 102.

In some embodiments, the device may comprise more than one processor 112. The processor 112 may comprise a computer-readable medium, such as a random access memory (RAM) 114 coupled to the processor. The processor 112 may execute computer-executable program instructions as described herein for analyzing the signals received from the sensor 104. The computer-executed instructions may be stored in memory 114.

The processor 112 may be configured to receive multiple electrical measures from a sensor 104. In other embodiments, the processor 112 may be configured to receive one or more electrical measures from multiple sensors. In the embodiment shown in FIG. 1, the sensor 104 and the processor 112 are in communication via a direct wired digital connection. Alternatively, the sensor may comprise fiber optic connections from the sensor or probe to downstream (e.g., amplifier, filter, etc.) components. In other embodiments, communication between a sensor 104 and the processor 112 may be through analog signals and may be wireless. For instance, the sensor 104 may be configured to use Bluetooth or Wi-Fi to communicate with the processor 112.

The processor 112 may be configured to transform the electrical measures using standard electrical signal analysis. For example, in certain embodiments, the processor 112 may transform the electrical signal received from the sensor to an electrical signal that can be correlated to a quantitative measurement. Also, the processor 112 may be configured to correlate the amplitude (or intensity) of the electrical signals to an output signal. In one embodiment, the processor 112 may transform the electrical signal received from the sensor 104 or sensors to a measurable signal or signals that can be correlated to an objective measure of pain.

Also, the processor may be configured to determine particular patterns of electrical signals that are correlated with pain or a physiological condition or ailment in a specific area of the body. For example, the processor may be configured to detect particular patterns of EMG signals that are correlated with pain in the spinal area, or with another medical condition or biological state. The processor may also be configured to filter the electrical signal(s) received from the sensor or sensors, such as, for example, to remove noise, if such filtering is not provided as a separate component.

The processor may receive and compare electrical signals collected during one use of the device. Alternatively, data from previous uses of the device may be stored in memory and used later. For example, when the processor 112 receives the electrical measure from the sensor 104, the processor may compare the measure to a previously-received electrical measure or to some other data stored in memory 114. For instance, the processor may be configured to determine the difference between a first electrical measure and a second electrical measure where the first electrical measure is stored in memory.

For example, in an embodiment, the processor may comprise a computer code for transforming the signal as received from the sensor, or a downstream amplifier, filter and/or monitor, to a signal that can be quantified and correlated to an objective measure of pain. For example, in an embodiment, the signal may be digitized. Additionally or alternatively, the signal may be compared to a control reading (e.g., a signal from a second sensor or a known baseline value) for removal of noise. In an embodiment, the signal may also be parsed into segments representing discrete electrical impulses or measurements. Additionally or alternatively, the signal may be normalized, undergo subtraction of the mean and/or transformed using other types of statistical-based analyses, which are well known to one of ordinary skill in the art.

In some embodiments the processor is also configured to accept data entry from other input devices 116. In one embodiment, input device 116 is a keyboard Input device 116 may be in the form of an input terminal (not shown). Alternatively, input may be accepted from other sources or devices, such as through the Internet or via removable media such as a memory card or floppy drive. Thus, the processor may be configured to adjust its calculations based on input data form other sources. For example, an operator may want to input variables or constraints for the analysis such as the threshold for determining noise. As another example, an operator may want to input a limit for determining the significance of changes in the muscle signals.

The processor 112 may be configured to generate one or more reports. A report, for example, may include historical data showing a trend line of muscle signals over time. Or, a report may comprise an analysis of the level of pain, the source of the pain, and a recommended treatment. The processor may output one or more reports to a printer (not shown). In another embodiment, the processor may output one or more reports over a network to a separate device, such as a personal digital assistant (PDA). The processor may also store one or more reports in the memory 114.

The device 102 may also comprise a diagnostic delivery device 118. The diagnostic delivery device 118 may be configured to deliver a therapeutic agent to a body part of the subject. In an embodiment, the diagnostic delivery device may comprise an access member. For example, the diagnostic delivery device may comprise a catheter. In another embodiment, the diagnostic delivery device may comprise a drug pump.

The therapeutic agent may be administered by a health care professional based on an evaluation of the measured electrical activity at the body part of interest. Or, in certain embodiments, the therapeutic agent may be automatically administered based on the level of the electrical activity measured at or near the body part. Thus, in certain embodiments, the device 102 may comprise a programmable dispenser.

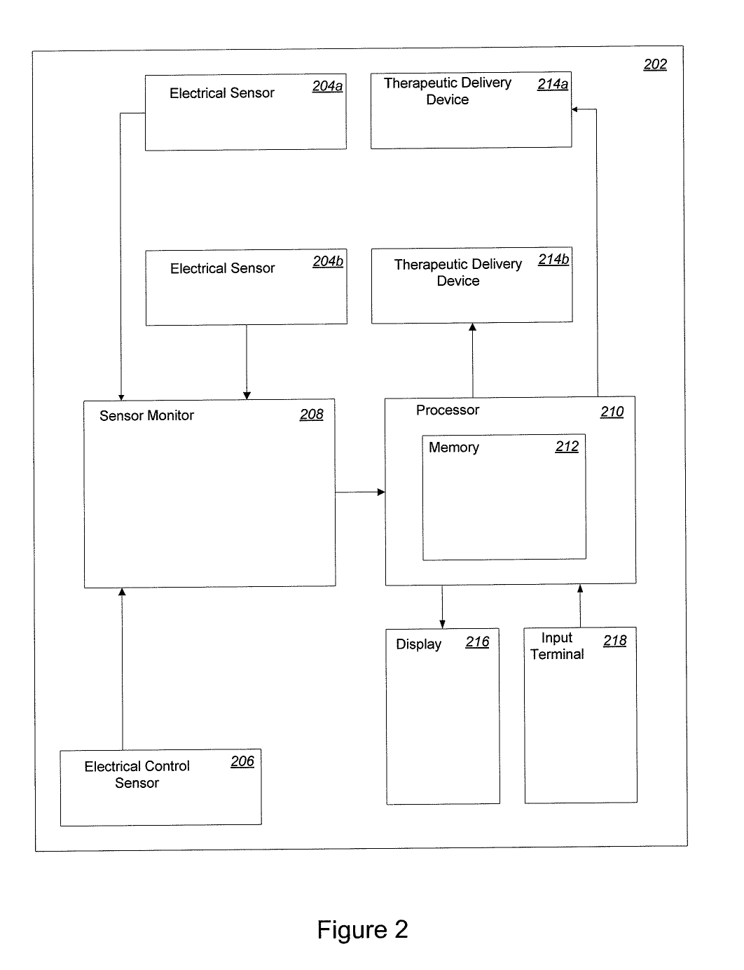

FIG. 2 is a block diagram illustrating an alternate embodiment of a device for diagnosing and treating medical conditions in the spine. In the embodiment shown, the device 202 comprises a plurality of electrical sensors 204a and 204b. Additional sensors (not shown) may be included (e.g., 204c, 204d, 204e). The plurality of electrical sensors 204 may be operable to measure the electrical activity at or near one or more body parts.

The plurality of electrical sensors 204 of the device 202 may be configured to detect electrical measures substantially simultaneously. For example, each electrical sensor 204 may be activated at the same time. Also, as described herein, in certain embodiments, at least one of the sensors may be an electrical control sensor 206. An electrical control sensor 206 may be positioned at or near a body part that is believed to be free of pain. Thus, in an embodiment, at least one electrical control sensor 206 may provide a baseline reading and/or negative control.

The plurality of electrical sensors 204 and 206 may transform the electrical signal to a signal that can be recognized by the apparatus. Thus, in an embodiment, the sensors (detectors) transform the electrical signal detected from the muscles or nerves near the body part, to an electrical signal that can be recognized by other components of the device. Thus, as described herein, the device 202 may further comprise an amplifier, a filter and/or a converter (not shown in FIG. 2).

In embodiments of the invention with a plurality of electrical sensors 204, the plurality of electrical sensors may be arranged so as to facilitate positioning at or near a body part of interest. In an embodiment, the sensors may comprise a substantially linear array. For example, such a linear array may be used for positioning a plurality of electrical sensors along the spine.

In one embodiment, a plurality of electrical sensors 204 may be used alongside a plurality of therapeutic delivery devices 214. For example, an array of EMG electrodes (e.g., 204a and 204b) may be implemented in combination with an array of catheters (e.g., 214a and 214b), each of which are inserted into one or more intervertebral discs. The catheters 214 may then be used to deliver substances into the discs. For example, traditional discography catheters and/or functional anesthetic discography techniques may be implemented in combination with methods described herein to diagnose the source of discogenic pain.

The plurality of electrical sensors 204 may be in communication with a sensor monitor 208. In one embodiment, the sensor monitor 208 is in communication with electrical sensor 204a and electrical sensor 204b to receive raw or transformed electrical signals form the sensors. For example, sensor monitor 208 may comprise an electromyography (EMG) monitor. Alternatively, sensor monitor 208 may comprise an electroencephalography (EEG) monitor. Or, other monitors known in the art of monitoring physiological (e.g., nerve and muscle) electrical activity may be used. For example, see U.S. Pat. No. 6,654,634, U.S. Pat. No. 6,306,100, U.S. Pat. No. 6,181,961, and U.S. Pat. No. 6,334,068 for multi-channel monitors that may be used with the apparatus and/or system of the present invention. The disclosure of U.S. Pat. Nos. 6,654,634, 6,306,100, 6,181,961, and 6,334,068 are incorporated by reference herein in their entireties for all purposes.

The device 202 may also comprise a processor 210. The processor 210 may be in communication with sensor monitor 208 to receive signals that are correlated to the electrical signals received by the sensors. In some embodiments of the present invention, the processor and the sensor monitor are combined. The processor may be configured to transform the electrical signals received from the sensors and/or monitor, or to correlate the signals to a quantitative measure of electrical activity and/or pain as described herein. Also, the processor may be configured to remove noise.

For example, in an embodiment, the processor 210 may comprise a computer code for transforming the signal as received from the sensor, or a downstream amplifier, filter and/or monitor, to a signal that can be quantified and correlated to an objective measure of pain. In an embodiment, the signal may be digitized. Additionally or alternatively, the signal may be compared to a control reading, such as a signal from a different sensor, for example the electrical control sensor 206 for removal of noise. In an embodiment, the signal may also be parsed into segments representing discrete electrical impulses or measurements. Additionally or alternatively, the signal may be normalized, undergo subtraction of the mean, and/or be transformed by other statistical-based analyses as described herein.

Also, the processor 210 may comprise a memory 212. For example in one embodiment, the memory 212 may be configured to store a plurality of muscle signals. The memory 212 may also be configured to store historical data, such as previous muscle signals received by the processor 210.

The device 202 shown may also comprise a display 216 in communication with the processor 210. The display may be configured to show graphical representations of the electrical measures or muscle signals received by the processor. In some embodiments, the display 216 may show reports comparing current muscle signals with muscle signals previously received by the processor 210. The report provided by the display 216 may be the same or similar to a paper report. Alternatively, the display 216 may comprise a means for a user to evaluate the report and modify the report as required (e.g., entry of identification variables (e.g., date or the patient's name) and/or entry of treatment recommendations based on the report. For example, the report may include historical data showing a trend line of muscle signals over time, an analysis of the level of pain, the source of the pain, and/or a recommended treatment.

The device 202 may further comprise an input terminal 218 in communication with the processor 210. The input terminal may comprise a keyboard or a computer configured to send data to the processor. For example, an operator may send input parameters to the processor relating to how the processor analyzes the muscle signals. The processor 210 may be configured to accept input from other sources, such as input received over a network (not shown).

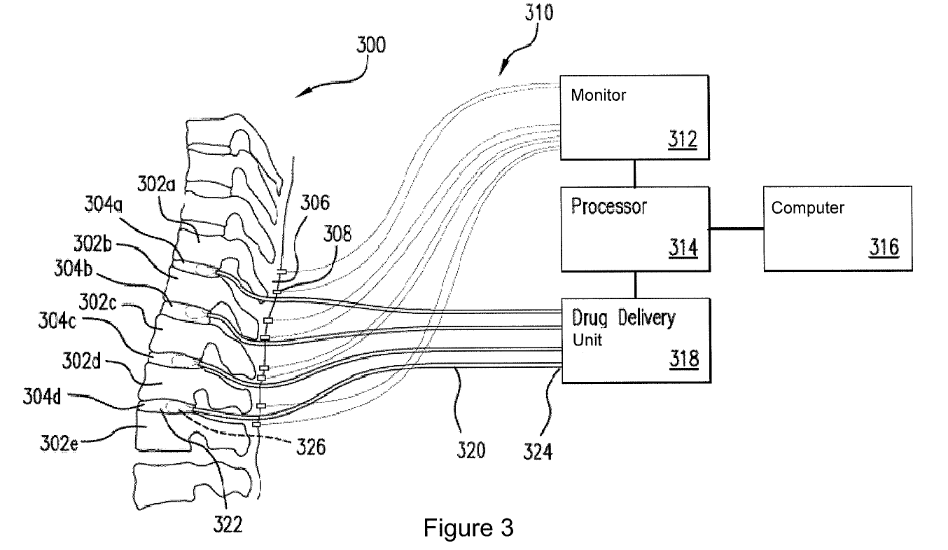

Certain embodiments of the present invention may comprise a feedback system in which signals from the body part of interest are used to determine whether a therapeutic agent should be administered. An embodiment of a feedback system of the present invention is shown in FIG. 3. As shown in FIG. 3, electrical probes may be positioned along the spine to detect electrical signals from muscles surrounding a plurality of intervertebral discs in the spine. The system may also include anesthetic delivery catheters placed at multiple levels of the spine for delivery of a therapeutic agent as required.

As shown in FIG. 3, the spine 300 is generally comprised of vertebral bodies (e.g., 302a, 302b, 302c, 302d and 302e) and intervertebral discs (e.g., 304a, 304b, 304c, and 304d). One or more layers of muscle 306 and other tissue are adjacent to the spine 300.

In the illustrated embodiment shown in FIG. 3, the system may comprise a sensor 308 or a plurality of sensors 308 for placement near a body part. For example, individual sensors 308 may be placed in or around the muscle 306 adjacent to the intervertebral discs 304a, 304b, 304c and 304d for at least a portion of the spine 300. In an embodiment, the sensors 308 are electrodes.

The sensors 308 may be in communication with a monitor 312. Monitor 312 may comprise an EMG monitor, an EEG monitor, or a nerve impulse monitor. In the case of an EMG monitor 312, EMG electrodes may comprise the individual sensors 308. An EEG monitor may be coupled or in communication with sensors 308 located on a subject's head. A catheter monitor may be in communication with one or more sensors or probes in one or more catheters anchored to a body part or body parts associated with pain. In the case of a pressure sensor, the monitor 312 may be configured to determine differentials in pressure. If, for example, an intervertebral disc exhibits changes in pressure while a subject initiates a routine or pain-inducing movement, it may be determined that the intervertebral disc is ruptured.

The monitor 312 may be configured to receive signals from the sensors 308. In one embodiment, the monitor 312 may quantify signals from the sensors. Also, as described herein, the sensors may be in communication with an amplifier, filter and/or converter as required (not shown in FIG. 3).

In an embodiment, the sensors' signals are transmitted by a wire 310 (e.g., a fiber optic connection). Alternatively, wireless transmission of the sensor signals may be used. Where remote transmission is used, the subject may not need to be in the same location as the detector. In an embodiment, monitor 312 analyzes the electrical signals generated by the sensor(s) 308.

The monitor 312 may be connected to a processor 314. The processor may be programmable or may be controlled by an computer 316. The input terminal may comprise a keyboard or a computer configured to send data to the processor.

The system may further comprise a drug delivery unit 318. In an embodiment, the drug delivery unit 318 is connected to the monitor 312 via the processor 314.

The drug delivery unit 318 may be configured to deliver a therapeutic agent to the body part of interest. For example, the drug delivery system may comprise an access member 320 (or a plurality of access members), such as a catheter, that can have the distal end 322 of the access member implanted in the body part(s) of interest 304 and the proximal end 324 accessible to a physician. In this way, the physician can inject a therapeutic agent into the disc(s) as required. Alternatively, drug delivery unit 318 may be a stomach pump or an insulin pump device.

In an example, where the system is set up to monitor electrical activity from four intervertebral discs (304a, 304b, 304c and 304d), it may be determined that only one of the discs (e.g., 304d) is exhibiting abnormal electrical activity. The access member 320 may comprise an expandable portion 326 (e.g. a balloon or spring-like material) at or near the distal end 322 of the catheter to facilitate implanting the catheter in the disc 304d in a secure manner. For example, the expandable portion may be inserted into an intervertebral disc in an unexpanded state and then be expanded in situ. In this way, it will be difficult to remove the distal end of the catheter from the disc, without putting the expandable portion back into an unexpanded state.

In one embodiment, the system shown in FIG. 3 may comprise an automatic feedback loop. In one example, the monitor 312 continuously monitors the signals received from the sensors 308. When the processor 314 determines that an objective level of pain reaches a predetermined level, the processor may activate the drug delivery unit 318 until the objective level of pain falls below the predetermined level. Further, the computer 316 may modify the automatic feedback loop by adjusting the predetermined level. The drug delivery unit 318 may be used in conjunction with other forms of medical intervention during an automatic feedback loop. For example, a feedback loop may further comprise medical intervention in the form of a spinal procedure intended to relieve pain. If the processor 312 determines that the spinal procedure successfully lowered a subject's objective level of pain below a threshold, than the processor may not activate the drug delivery unit 318.

Thus, in certain embodiments, the physician may use the drug delivery unit 318 to inject a therapeutic agent (e.g., pain killer or other drug) to treat the intervertebral disc that is causing the patient a high amount of pain as measured using the objective monitors of the system. One of ordinary skill in the art having the benefit of this disclosure would appreciate that various configurations of sensors and monitors may be used. Some examples of monitoring apparatus are described in U.S. Pat. Nos. 6,654,632, 6,654,634, 6,306,100, 6,181,961 and 6,334,068.

In another embodiment, the system shown in FIG. 3 may be configured as an automated or semi-automated system for identifying specific spinal level that is causing spinal pain by systematically treating one level after the next within the spinal column with anesthetic or other medication, while continuously monitoring electrical signals from the spine, in order to locate the particular level that is causing the spinal pain. Additionally or alternatively, the system may comprise an override and/or warning if it appears that regulation of delivery of the therapeutic agent is required.

Embodiments of the present invention also comprise methods for the diagnosis and/or treatment of pain. In some embodiments, the present invention may provide a method to objectively quantify pain. The system may also comprise a device for a subject to indicate a subjective level of pain. As an example, computer 316 may be coupled to a dial (not shown). During the feedback process, the subject may indicate a level of pain by rotating the dial to a certain level.

Also, the methods of present invention may be utilized to diagnose the source of pain. For example, once the objective level of pain has been determined, the area may be tested to determine if a medical intervention reduces the objective level of pain. If a medical intervention, such as application of an analgesic to a specific location on the spine, does not relieve any pain or change the muscle signals, than that specific location may be eliminated as the potential source of the pain.

In yet other embodiments, the present invention provides methods of treatment. In one example of a method for diagnosing and treating pain, the processor may be configured to detect particular patterns of EMG signals that are correlated with pain in the spinal area. When an EMG signal pattern suggests that the subject is experiencing pain, the drug pump may be activated to deliver medication to suppress the pain in the spine.

In some embodiments, the methods of the present invention may comprise a feedback loop for treating a subject. As an example, when a subject initially experiences pain, the determination of whether the subject requires medical intervention may be made. Following the medical intervention, the subject may then be under continued observation to determine how much pain was alleviated by the medical intervention, and if additional medical intervention is needed. In such an example, an operator may be able to determine the efficacy of certain medical interventions according to specific conditions. Such conditions may include, for example, the type of pain, the location of the pain, and other characteristics related to the subject. Additionally or alternatively, the method may utilize a processor configured to increase the amount of medication being infused into the spine when the intensity of the detected EMG signal is increasing. Alternatively, the system may be configured to continuously deliver a based-line level of analgesic, and/or to decrease the level of analgesic being infused depending on the detected EMG signal.

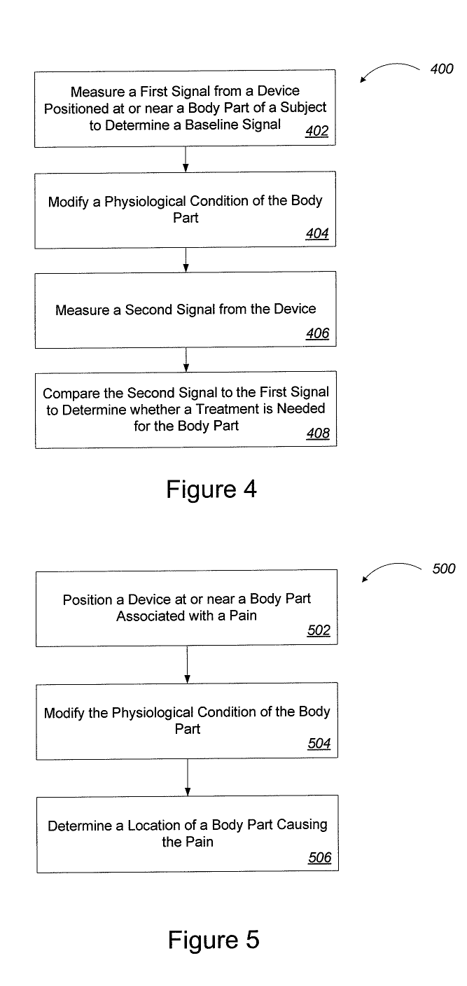

FIG. 4 is a flow diagram illustrating a first method for the diagnosis and treatment of medical conditions in a body part in accordance with one embodiment of the present invention. As shown in FIG. 4, the first step in method 400 comprises measuring a first signal from a device positioned at or near a body part of a subject to determine a baseline signal 402. In one variation, the device is configured to measure or monitor the physiological condition of a body part. Such a device may be a probe, sensor (e.g. pressure sensor or electrical sensor), conduction pads, or a transducer. For example, a pressure sensor may measure or monitor the pressure inside an organ or body part.

The device may be in the form of electrodes consisting of a pad or pads, or a needle or needles. The electrical activity picked up by the electrodes may be displayed on an oscilloscope, monitor, or display. Optionally, an audio-amplifier is used so that the activity can be heard. The electrical measure may comprise an electrical measure on any suitable scale, such as millivolts or microvolts.

Muscle tissue may produce little or no electrical activity during rest. When an electrode is inserted, a brief period of activity may be seen on the oscilloscope, but after that, little to no signal should be present. Thus, a baseline signal may consist of the absence of a signal, or a null reading.

The device may be anchored at or near the body part. For example, the device may comprise a member configured to expand at or the body part, such as a balloon, a grapple, or a hook. In other embodiments, other methods of anchoring the device to the body part may be utilized.

Step 404 comprises the step of modifying a physiological condition of the body part. For example, a drug delivery pump may deliver an anesthetic or an analgesic to the body part. Alternatively, a surgical procedure may be performed on the body part.

Step 406 comprises the step of measuring a second signal from the device. The device may remain in the same position during steps 402, 404, and 406. For example, a catheter may be anchored to an intervertebral disc during steps 402, 404, and 406. The second signal may be measured by the device while a subject performs an activity normally associated with pain. For example, a patient may want to discover the location of a spinal pain. After a physiological condition of a specific area of the spine thought to be the source of the pain is modified, the patient is asked to repeat a movement or activity which normally causes the patient pain. A second signal is then measured 406 while the patient is repeating the activity.

Step 408 comprises the step of comparing the second signal to the first signal to determine whether a treatment is needed for the body part. Treatment may be appropriate where the second signal and the first signal are different. In such an example, a difference between the second signal and the first signal may indicate that the modification in the physiological condition of the body part 404 was successful in reducing pain. Treatment may not be appropriate where the second signal and the first signal are the same. In such an example, a lack of difference between the second signal and the first signal may indicate that the modification in the physiological condition of the body part 404 did not accomplish a reduction in pain.

In one example, a baseline signal is detected by a sensor anchored in an intervertebral disc 402. Next, an anesthetic is introduced into the intervertebral disc 404. After the anesthetic is introduced 404, a second signal or reading is detected by the sensor. If the sensor detects a second signal which is different than the first baseline signal, than it may be determined that treatment is needed for the intervertebral disc. If the sensor detects a second signal which is not different than the first baseline signal, than it may be determined that no treatment is needed for that specific intervertebral disc. In such a scenario, a sensor may be placed at another, adjacent body part, to determine through an iterative process the source of pain.

FIG. 5 is a flow diagram illustrating a second method for the diagnosis and treatment of medical conditions in a body part in accordance with one embodiment of the present invention. As shown in FIG. 5, the first step in method 500 comprises positioning a device at or near a body part associated with a pain, the device configured to modify a physiological condition of the body part 502. Such a device may be a drug delivery device, drug pump, transducer, or a heat element. In another variation, the device may be further configured to measure the physiological condition in a body part. Such a device may be a probe, sensor (e.g. pressure sensor or electrical sensor), conduction pads, or a transducer.

The device is positioned at or near a body part causing pain to the subject. For example, where a subject is experiencing spinal pain, one or more sensors may be placed on the skin nearest the spine, or inserted through the skin into the back muscles of the patient. While FIG. 3 shows a thoracic spinal region, embodiments of the invention may be used in various parts of the body.

The sensors may be in the form of electrodes consisting of a pad or pads, or a needle or needles. The electrical activity picked up by the electrodes may be displayed on an oscilloscope, monitor, or display. Optionally, an audio-amplifier is used so that the activity can be heard. The electrical measure may comprise an electrical measure on any suitable scale, such as millivolts or microvolts.

Muscle tissue may produce little or no electrical activity during rest. When an electrode is inserted, a brief period of activity may be seen on the oscilloscope, but after that, little to no signal should be present.

Once the sensor has been placed at or near a body part 502, the method 500 further comprises modifying the physiological condition of the body part 504. For example, an anesthetic or an analgesic may be introduced to the body part. Alternatively, pressure, heat, or electricity may be introduced to the body part by a device.

Next, method 500 comprises determining a location of a body part causing the pain based at least in part on a physical response to the physiological modification 506. The physical response to a physiological modification may be a lack of pain. For example, if the subject repeats an activity normally associated with pain, the physical response to a physiological modification may be a lack of pain. Alternatively, a physical response may comprise pain. For example, if the location of the pain is not physiologically modified, than there may be no physical response. In such a variation, although a location of the body part causing the pain has not been located 506, a location of the body part not causing the pain has been ruled out. By engaging in an iterative process, locations not causing pain may be eliminated one by one, or in parallel, until the body part causing the pain has been determined.

In one embodiment of the method, after all of the electrodes have been inserted near a muscle 502, the subject patient may be asked to reposition the body part which intersects with a nearby muscle. For example, to make an assessment of the spine, the subject may be asked to lift a leg or twist from the waist. When the subject is asked to move his or her spine, for example, in motions that typically cause the subject spinal pain, one or more muscle signals may be generated by the electromyography indicating the electrical activity level of the subject's spine. The action potential (size and shape of the wave) created by the electrical signals and displayed on the oscilloscope may provide information about the ability of the muscle to respond when the nerves are stimulated. As the muscle contracts more forcefully, more and more muscle fibers may be activated, producing action potentials.

A healthy muscle may show no electrical activity (no signs of action potentials) during rest, only when it contracts. However, if the muscle is damaged or has lost input from nerves, it may have excess or abnormal electrical activity during rest. Also, if the nearby nerve is damaged (e.g., due to a compressed disc) there may be abnormal electrical activity even when the muscle is at rest. Alternatively or additionally, when the muscles or nearby nerves are damaged, electrical activity that results after contraction of the muscle or stimulation of the nerve may produce abnormal patterns.

The electrical measurements may be taken from the sensors during normal daily activity, or when performing a functional motion that mimics pain. The measurements may be made in the physician's office. Alternatively, where the sensors employ wireless transmission, the measurements may be made in a location (e.g., at home or at work) that is remote from the location of the monitoring device (e.g., doctor's office). The measurements may be processed and displayed on a monitor, recorded in the memory of a computer, or transmitted to a printer.

The electrical measurements may be compared to other electrical measurements. For example, an operator may compare the measurements to a set of known pain readings from other patients.

Alternatively, the muscle signal may be received by a sensor, and then the signal compiled by a processor. The processor may compare the current measurements with the patient's historical readings. In one embodiment, the electrical measurements of one body part under pain may be compared with the electrical measurements of another body not under pain.

After an electrical level at or near a body part has been measured, the measurement may be used to determine an objective level of pain. In one embodiment, an objective score for the pain may be determined after the electrical measurements are correlated to standard values or to readings previously made from the same patient.

In some cases the method may comprise the subject making a subjective rating of the pain. In some embodiments, the subject may rate the pain at the same time or before an objective level of pain is determined. A subject's subjective level of pain may be correlated with the objective level of pain. Correlating a subjective level of pain with an objective level of pain may, for instance, assist a physician in diagnosing the pain.

An embodiment may additionally comprise generating a report. In one embodiment, a processor 112 may generate one or more reports. A report, for example, may include historical data showing a trend line of muscle signals over time. Or, a report may comprise an analysis of the level of pain, the source of the pain, and a recommended treatment. A report may be generated on a display 218, or generated to some other device such as a printer.

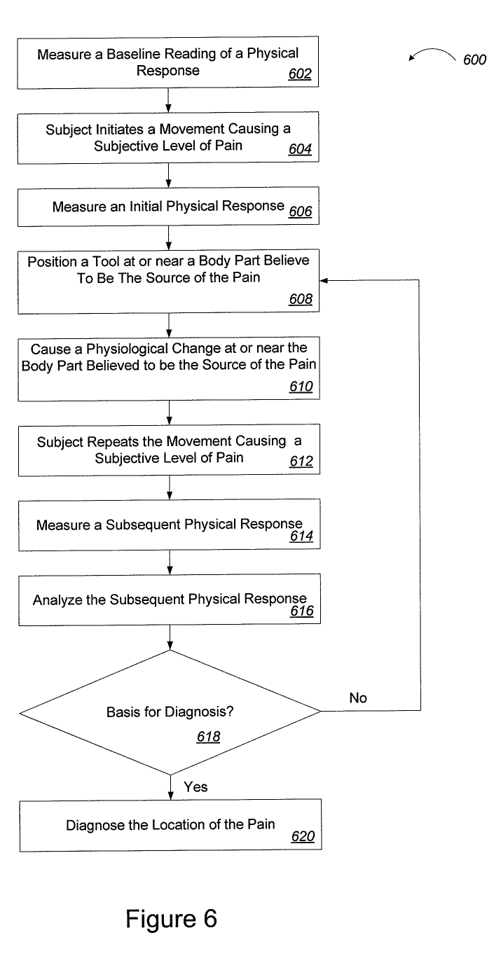

FIG. 6 is a flow diagram illustrating a third method for the diagnosis and treatment of medical conditions in a body part in accordance with another embodiment of the present invention. As shown in FIG. 6, the method may begin by measuring a baseline reading of a physical response 602. As an example, the pressure in an intervertebral disc may be measured while a subject is at rest. The baseline reading may be at or near a body part believed to be the source of the pain. Alternatively, a baseline reading may be taken from an EEG sensor on a subject's head, or an EMG electrode placed on a muscle or muscles of the subject. The baseline reading may normally be taken when the subject is at rest, or not experiencing pain. The baseline reading may be a control reading which may assist subsequent evaluation. For example, a control signal may indicate a normal level of electrical noise detected by a sensor which is not indicative of pain. The subject may be asked to subjectively provide a baseline reading, for example to confirm that the subject is not experiencing pain while at rest.

After the baseline reading is taken 602, the subject may initiate a movement which causes the subject a subjective level of pain 604. For example, a physician may ask a patient to assume a position, make a movement, or otherwise induce a physical change which is known to cause the subject pain.

During or after the subject initiates a movement causing pain, 604, an initial physical response may be measured 606. One or more sensors 204 may be placed at or near the body part believed to be causing the subject pain. The subject's movement may generate an electrical signal detected by a sensor 204a, which may transmit a signal to the processor 112. In another embodiment, a pressure sensor located inside a body part may detect a change in pressure as the physical response to the movement. At this point in the procedure, it may be possible to determine the location of pain based on the difference between the initial physical response 606 and the baseline reading 602. In some instances, measuring a baseline reading 602 and/or measuring a physical response 606 may not be performed.

After performing some, all, or none of steps 502, 504, or 506, a tool is positioned at or near a body part believed to be the source of the pain 608. As one example, a catheter may be positioned at nor near an intervertebral disc. In other examples, a drug pump may be positioned at or near an organ or muscle.

After the tool is positioned 608, the tool causes a physiological change at or near the body part believed to be the source of the pain 610. For example, a drug pump may deliver insulin or an analgesic to a body part. Specifically, a physician may anesthetize a specific area or body part thought to be the source of the pain. In other examples, a probe may be configured to generate vibrations, heat, electrical pulses, or microwave radiation.

After a physiological change is caused at or near the body part believed to be the source of the pain 610, the subject is asked to repeat a pain-causing movement, or induce a change in the body part associated with pain 612. As one example, five to ten minutes after a body part has been anesthetized, a subject is instructed to perform the same pain-eliciting activities performed before the physiological change 610.

The pain-generating movement 612 may cause a subsequent physical response. For example, an intervertebral disc may lose pressure, or a subject's brain may generate electrical signals associated with pain. The subsequent physical response is then measured 614. As one example, a catheter sensor may detect a change in pressure in an intervertebral disc. As another example, an electrical signal detected through EMG may be measured.

After the subsequent physical response is measured 614, the subsequent physical response is analyzed 616, and optionally compared with the baseline physical response. In one embodiment, the initial physical response measured before the physiological change is compared with the subsequent physical response measured after the physiological change.

Analyzing the subsequent physical response 616 may lead to a basis for diagnosis 618. For example, if the subsequent physical response is much less severe than the initial physical response, there may be a basis for diagnosis. Any difference between the initial physical response 606 and the subsequent physical response 616 may have been a result of the physiological change at or near the body part believed to be the source of the pain 610. Alternatively, if the initial physical response and the subsequent physical response are the same, than it may be determined that there is no basis for diagnosis. For example, if an analgesic is injected into a disc which is not the source of the pain, the pain may persist and a subsequent EMG signal (and the pain) would not be suppressed.

In some instances, the subsequent physical response 614 may be analyzed without measuring an initial physical response 606.

It there is a basis for diagnosis 618, a location of the pain may be diagnosed 620. If the physiological change is believed to have caused a change or reduction in physical response, than the body part where the physiological change was made may be diagnosed as the location of the pain.

If there is no basis for diagnosis, another iteration may be performed. Specifically, the tool may be repositioned at or near a second body part believed to be the source of the pain 608. Alternatively, multiple tools may have been pre-positioned before making any physiological changes. In the case of multiple tools, if there is no basis for diagnosis 618 after a first physiological change, a second physiological change may be made by a second tool already in position at a second body part, without repositioning the first tool.

In one example, a subject reports feeling spinal pain in a generalized location, such as the lower back, while she performs a calf stretch. A physician may position and anchor a tool, such as a catheter, at an individual intervertebral disc believed or guessed as the source of pain 608. The tool then causes a physiological change at the individual intervertebral disc 610, for example by delivering an anesthetic to the disc lumen.

After the physiological change, the subject repeats the calf stretch which caused the initial pain. A subsequent physical response correlated with the stretch is then measured 614. If the subsequent physical response, such as pain, no longer exists, than there may be a basis for diagnosis 518, and the location of the pain may be diagnosed 620 as the body part where the tool was initially positioned, and the physiological change was made. If the subsequent physical response 514 did not change from the initial physical response 606, for example, the subject still experiences pain, than there may not be a basis for diagnosis 618. A physician may then reposition the tool at a second body part believed to be the source of the pain, and the process is repeated.

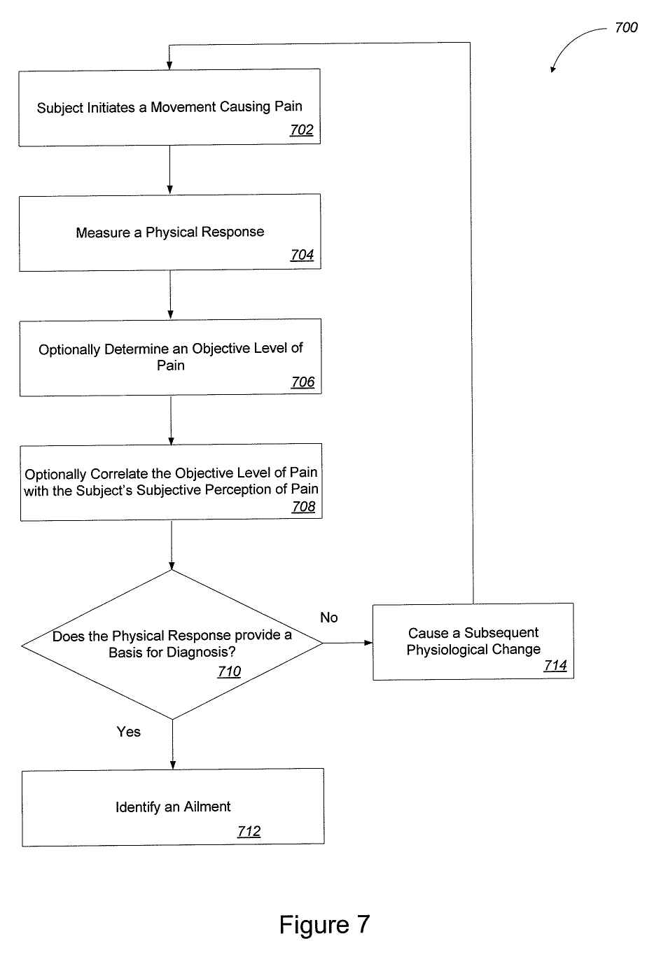

FIG. 7 is a flow diagram illustrating a fourth method for the diagnosis and treatment of medical conditions in a body part in accordance with another embodiment of the present invention. In an embodiment, one or several steps of method 700 may be automated. In yet other embodiments, the method 700 may comprise a feedback loop.

In step 702, a subject initiates a movement causing a subjective level of pain with the subject. For example, a physician may instruct a patient to perform or repeat a task or assume a position that induces pain in the spinal region.

The subject's movement to induce pain may generate a physical response which is measured by the processor 704. The physical response immediately following the subject's movement may directly correlate with a subject's pain.

In step 706, an objective level of pain is optionally determined. In one embodiment, the level of the electrical measurement is compared with previously-recorded electrical measurements from the same body part to determine if the objective level of pain is worse or better than in the past.

After an objective level of pain is determined 706, the optional step may be taken of correlating the objective level of pain with the subject's subjective level of pain 708. In one embodiment, the subject is asked to rate the sensation of pain using a predefined scale, such as the visual analog scale. The subjective ratings provided by a patient may be used in combination with the muscle signals to diagnose the location causing the pain.

Next, method 700 determines whether the physical response provides a basis for diagnosis 710. For example, if a new physical response is markedly different than an original physical response, than the location of pain may be determined. As another example, if a differential between a first physical response and a second physical response exceeds a threshold, than there may be a basis for diagnosis.

Once a source of pain has been diagnosed 712, an ailment may be identified 714. For example, a specific intervertebral disc may be diagnosed as ruptured.

If the physical response does not provide a basis for diagnosis 710, than a subsequent physiological change may be made 714. As one example, a processor may generate a signal which causes a drug delivery device to pump insulin into the liver. As another example, a treatment signal may cause a pre-positioned catheter to deliver an anesthetic to a different location.

After the physiological change has been caused 714, the subject may be asked to re-initiate the same movement which previously caused pain 702 in order to evaluate the efficacy of the medical intervention or physiological change 714. For example, the subject may be asked to repeat the same movement earlier performed which generated a muscle signal. By comparing the previously determined objective level of pain with a new objective level of pain, the effectiveness of a medical intervention may be evaluated.

In one embodiment, method 700 comprises an automated feedback loop. For example, a processor may continually monitor a subject's physical responses to physiological changes, and automatically adjust the delivery of a therapeutic agent based on the latest physical response. During a feedback loop, the subject's physiological state may be automatically or manually changed. For example, a processor may automatically deliver a therapeutic agent, such as an analgesic, to the subject. Alternatively, the change in the physiological state may comprise a different medical intervention, such as surgery.

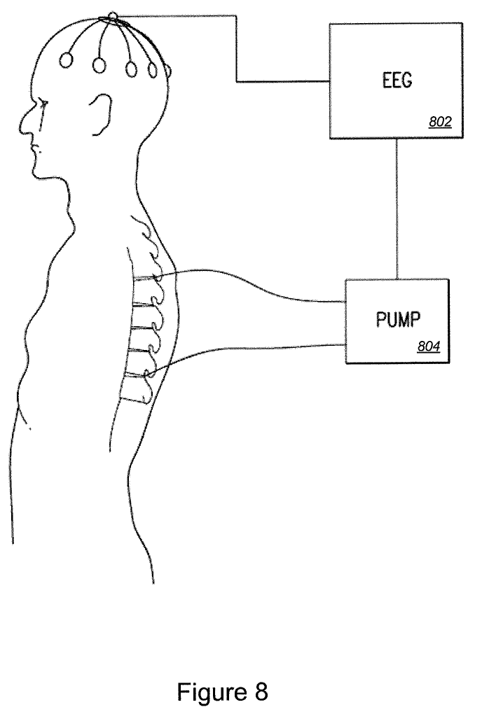

FIG. 8 is an illustration of a second configuration for diagnosing and/or treating spinal pain in accordance with one embodiment of the present invention, where a drug delivery device (e.g. a plurality of catheters having deployable anchors, etc.) is placed at multiple levels of the spine and EEG probes are positioned along the head. The system illustrated in FIG. 8 comprises an EEG 802. One or more EEG electrodes may be attached to a subject's scalp, and may be configured to detect electrical activity level. In other embodiments, other devices may be used to detect activity levels in a subject.

The system shown in FIG. 8 further comprises a drug delivery system 804, which may be configured to deliver an anesthetic or an analgesic to a subject. In one example, the drug delivery system 804 may comprise one or more catheters anchored to the subject's spine. The drug delivery system 804 can comprise one or a series of drug pumps. The drug pumps may be used to selectively anesthetize individual intervertebral discs.

In an example, a tool configured to cause a physiological change, such as a drug delivery device 804, may be positioned at or near an intervertebral disc associated with a level of pain in a subject. The drug delivery device may then cause a physiological change at the intervertebral disc, by delivering an anesthetic. Next, the subject may be asked to induce a change in the intervertebral disc, such as stretching or flexing, which typically generates pain.

If the EEG 802 does not detect an electrical level associated with pain after the physiological change, the location of the pain may be diagnosed. If the EEG 802 continues to detect electrical levels associated with pain, an additional physiological change may be caused at a successive intervertebral disc. Through this iterative process of selective anesthetization, the location of intervertebral pain may be diagnosed.

As an example, an EEG 802 may measure electrical activity produced by a subject's brain. If the EEG detects electrical activity indicative of pain, the EEG may generate a signal that causes the drug delivery device to anesthetize an intervertebral disc.

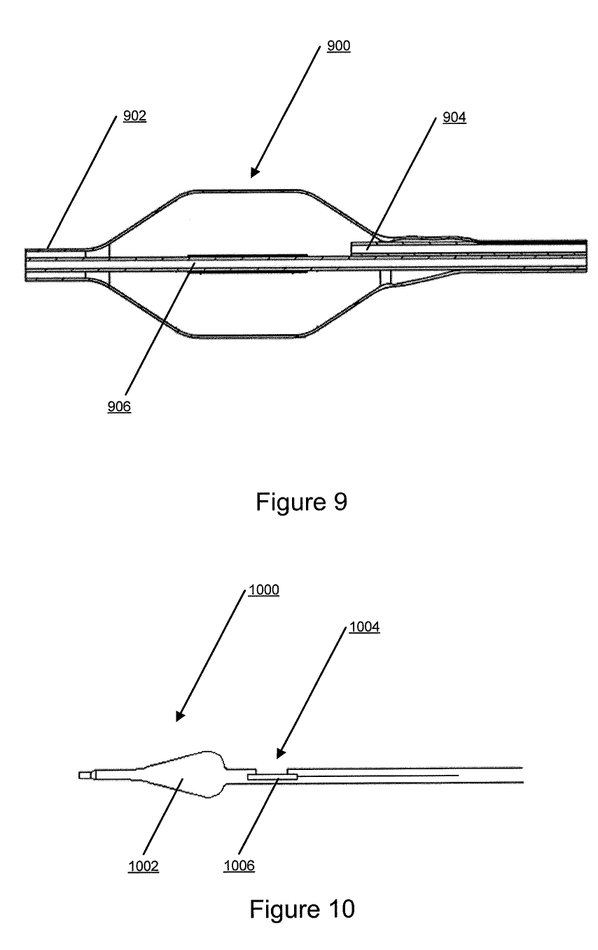

FIG. 9 is a first illustration of a device for diagnosing and/or treating spinal pain in accordance with one embodiment of the present invention. In the illustrated embodiment shown in FIG. 9, the device for diagnosing and/or treating spinal pain comprises a balloon catheter 900. A balloon catheter device 900, such as a Kyphon Discyphor™ Catheter System, may be anchored to an intervertebral disc.

The balloon catheter 900 may comprise an electrically conductive surface or probe 902. The balloon catheter 900 may further comprise an inflation lumen 904. Radiopaque contrast may be slowly injected into the balloon catheter 900 through the inflation lumen 904.

The balloon catheter 900 may also comprise an inner lumen 906. Various substances or devices may be introduced into a cavity that the balloon catheter 900 is anchored to through the inner lumen 906. For example, a guide wire may be introduced into an intervertebral disc cavity through the inner lumen 906. Alternatively, an anesthetic or an analgesic may be introduced into a body part cavity through the inner lumen 906.

FIG. 10 is a second illustration of a device for diagnosing and/or treating spinal pain in accordance with one embodiment of the present invention. In the illustrated embodiment shown in FIG. 10, the device for diagnosing and/or treating spinal pain comprises a catheter 1000. The catheter 1000 may comprise an anchor 1002. Anchor 1002 may be configured to inflate and deflate, like a balloon. As shown in FIG. 10, the anchor 1002 is deployed or inflated. Various substances may be used to inflate the anchor 1002, such as a radiopaque contrast or saline solution. In other embodiments, the anchor 1002 is deployed through other means, such as a differential in pressure.

The catheter 1000 may comprise an optional opening 1004. A probe 1006 may be coupled to the optional opening 1004. The probe 1006 may comprise an electrical probe, electrical sensor, or a motion transducer. Probe 1006 may be operable to detect an electrical signal, pressure, or motion. The probe 1006 may be able to provide more active feedback, or a higher resolution for detection than other devices, such as an EEG. As one example, the catheter 1000 may be inserted into an intervertebral disc of a subject. As the subject flexes, or otherwise attempts to induce pain, the probe 1006 may measure changes in pressure in the intervertebral disc, or measure electrical signals generated local to the intervertebral disc. The probe may comprise a pressure sensor (e.g. a semiconductor pressure sensor manufactured with micromachining technology) for detecting pressure changes in the body part (e.g. an intervertebral disc).

Alternatively, probe 1006 may be configured to induce a physiological change. For example, probe 1006 may be a motion transducer, configured to vibrate. Probe 1006 may alternatively be configured to produce heat, pressure differentials, vibration, or microwave radiation.

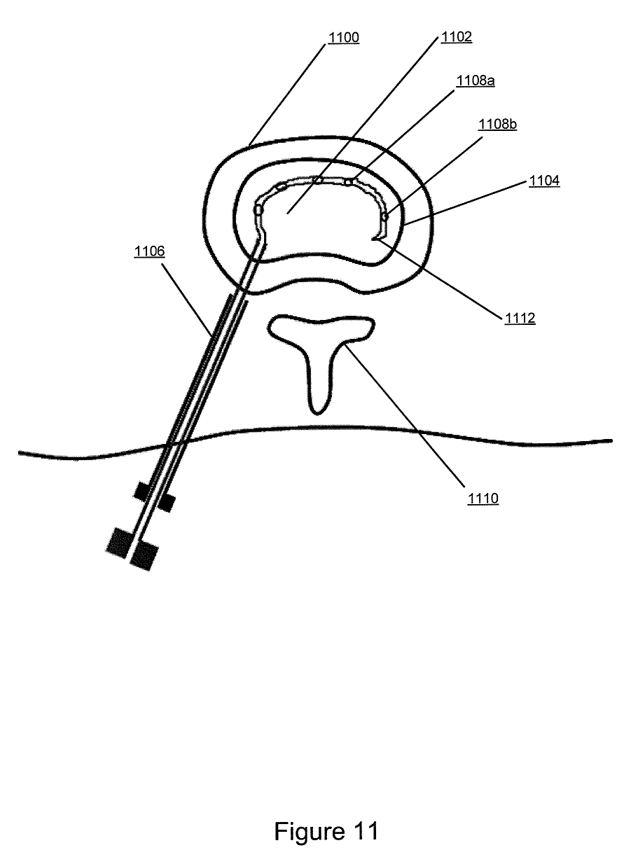

FIG. 11 is an illustration of a third device for diagnosing and/or treating spinal pain in accordance with one embodiment of the present invention. FIG. 11 illustrates a spinal lamina 1110 and an intervertebral disc 1100. The intervertebral disc 1100 further comprises a nucleus 1102 surrounded by an annulus 1104.

In the illustrated embodiment shown in FIG. 11, the system may comprise an access member 1106. In one variation, the access member may be an elongated member, comprising a hollow cylinder, tube, cannula, or a delivery catheter.

Optionally, the access member 1106 may be anchored or docked. In one embodiment, the access member 1106 is anchored to the intervertebral disc. The access member 1106 may be tightly anchored or loosely anchored to various places. The access member 1106 may be anchored through an inflatable, flexible balloon, or a more rigid, whisk-like anchor capable of being fully or partly retracted.

By anchoring the access member 1106, a subject may be able to perform activities that typically generate pain. For example, one or more access members 1106 may be anchored to a subject's spine. Next, the subject may perform activities, such as stretching or flexing, that typically generate pain, while the access members 1106 remain anchored to the intervertebral disc.

The access member 1106 may be positioned directly on the intervertebral disc, or slightly inside the outer fibers of the annulus of the intervertebral disc. The access member 1106 may be positioned using fluoroscopic guidance. It may be advantageous to ensure the access member 1106 is firmly docked to the intervertebral disc.

Multiple access members 1106 may be docked to multiple intervertebral discs 1100 at the same time. Multiple catheters may be used to isolate and identify the source or location of a subject's spinal pain. By anchoring multiple catheters, a group of intervertebral discs suspected of generating pain may be selectively anesthetized while the subject performs activities that typically generate pain.

FIG. 11 illustrates one embodiment where the access member comprises a plurality of sensors (e.g. 1108a and 1108b) or probes distributed along the length of the access member 1106, for detecting physiological conditions. The sensors may be attached to a flexible, semi-rigid, or rigid member 1112. Member 1112, may be, for example, a nano-sized tube capable of being threaded around the intervertebral cavity 1102.

In another variation, the access member is configured such that devices or substances may be inserted through a lumen in the access member into the body part. In one example, a radiopaque contrast is injected through the lumen of the access member to perform provocative discography. As another example, the spinal needle may be used to deliver a guide wire to the intervertebral disc lumen.

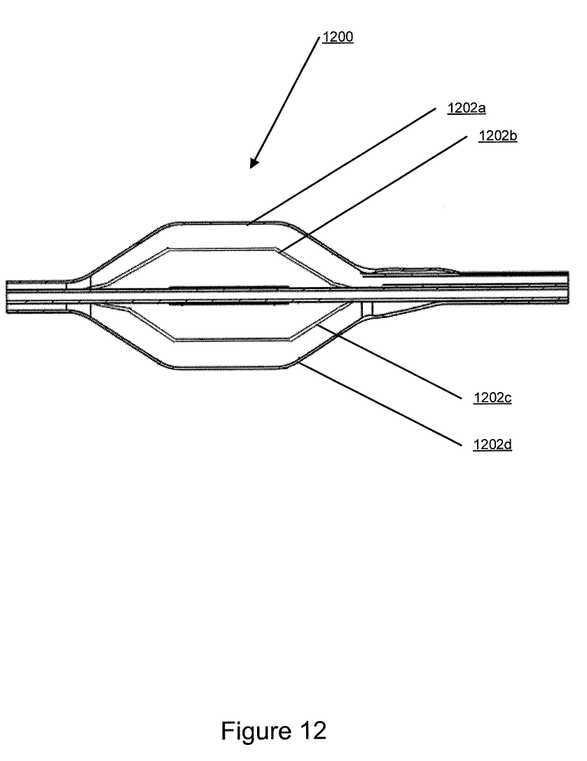

FIG. 12 is a fourth illustration of a device for diagnosing and/or treating spinal pain in accordance with one embodiment of the present invention. In the illustrated embodiment shown in FIG. 12, the device 1200 for diagnosing and/or treating spinal pain comprises one or more expandable members 1202a, 1202b, 1202c, 1202d. The members may be expanded by remote operation, or at the proximal end of the device. The members may comprise a rigid or semi-rigid material. As an illustration, the members may appear similar to a cooking whisk. The device may comprise other methods for anchoring to a body part, such as a grapple, or retractable hooks.

It will be understood that each of the elements described above, or two or more together, may also find utility in applications differing from the types described. While the invention has been illustrated and described as methods, systems, and computer-readable media for the diagnosis and treatment of medical conditions in the spine, it is not intended to be limited to the details shown, since one or ordinary skill in the art having the benefit of this disclosure would appreciate that various modifications and substitutions can be made without departing in any way from the spirit of the present invention. Where method and steps describe above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. As such, further modifications and equivalents of the invention herein disclosed may occur to persons skilled in the art using no more than routine experimentation, and all such modifications and equivalents are believed to be within the spirit and scope of the invention as described herein. All patents and published patent applications referred to in this document are incorporated by reference in their entireties as if each individual publication or patent application were specifically and individually put forth herein.

That which is claimed is:

1. A method comprising:
measuring a first signal from a device positioned at or near a body part of a subject to determine a baseline signal;
modifying a physiological condition of the body part;
measuring a second signal from the device;
comparing the second signal to the first signal to determine whether a treatment is needed for the body part.

2. The method according to claim 1, further comprising positioning the device at or near the body part.

3. The method according to claim 2, further comprising anchoring the device at or near the body part.

4. The method according to claim 1, wherein the body part is associated with pain.

5. The method according to claim 1, wherein the body part comprises a portion of the patient's spine.

6. The method according to claim 1, wherein the body part comprises an intervertebral disc.

7. The method according to claim 1, wherein the body part comprises at least an intervertebral disc and an adjacent vertebral body.

8. The method according to claim 1, wherein the body part comprises a vertebral body.

9. The method according to claim 1, wherein modifying the physiological condition of the body part comprises implanting a device into the body part.

10. The method according to claim 1, wherein modifying the physiological condition of the body part comprises modifying or altering a tissue in the body part.

11. The method according to claim 1, wherein modifying the physiological condition of the body part comprises injecting a substance into the body part.

12. The method according to claim 11, wherein the substance comprises an anesthetic an analgesic, or a radiopaque marker.

13. The method according to claim 11, wherein the substance is injected through a catheter having its distal end anchored in the body part.

14. The method according to claim 1, wherein the first signal and the second signal comprise EMG signals.

15. The method according to claim 1, wherein the first signal and the second signal comprise EEG signals.

16. The method according to claim 1, wherein measuring a first signal from a device comprises measuring the first signal from a plurality of detectors each configured to measure a different physiological parameter; and
measuring a second signal from the detector comprises measuring the second signal from the plurality of detectors.

17. A method comprising:
positioning a device at or near a body part associated with a pain, the device configured to modify a physiological condition of the body part;
modifying the physiological condition of the body part; and
determining a location of a body part causing the pain based at least in part on a physical response to the physiological modification.

18. The method of claim 17, further comprising:
measuring a first signal corresponding to a physiological parameter before modifying the physiological condition of the body part;
measuring a second signal corresponding to the physiological parameter after modifying the physiological condition of the body part; and
determining a comparison comparing the first signal with the second signal, and wherein the determining a location causing the pain is based at least in part on the comparison.

19. The method of claim 18, wherein the physiological parameter comprises pain.

20. The method of claim 17, wherein determining the location of the body part causing the pain comprises causing a movement associated with the pain.

21. The method of claim 17, further comprising:
repositioning the tool at or near a second body part associated with pain,
modifying the physiological condition of the second body part;
determining a location of a body part causing the pain based at least in part on a physical response to the physiological modification of the second body part.

22. The method of claim 17, wherein the tool comprises a catheter.

23. The method of claim 17, wherein the tool is configured to be anchored at or near the body part associated with a pain.

24. A method comprising:
positioning a first tool at or near a first body part associated with pain, the first tool configured to modify a physiological condition of the first body part;
positioning a second tool at or near a second body part associated with pain, the second tool configured to modify a physiological condition of the second body part;
modifying the physiological condition of the first body part;
modifying the physiological condition of the second body part; and
determining a location of the pain based at least in part on a physical response to the first physiological modification and a physical response to the second physiological modification.

25. A method to diagnose a location of pain in a subject comprising:
measuring a baseline level of a pain;
anchoring a diagnostic catheter within a space of a spinal disc, the spinal disc associated with the pain, the diagnostic catheter configured to cause a physiological change at or near the portion of the body part;
modifying a physiological condition of the body part;
causing a repetition of a physical activity associated with the pain;
measuring a subsequent level of the pain;
determining a differential between the baseline level of the pain and the subsequent level of the pain; and
diagnosing the location of the pain based at least in part on the differential between the baseline level of pain and the subsequent level of pain.

26. An apparatus comprising:
a device configured to detect or modify a physiological condition of a body part, the body part believed to be causing, or associated with, the generation of pain; and
a processor in communication with the device, the processor configured to determine a location of a body part actually causing the pain based at least in part on a physical response to a modification of the physiological condition of the body part.

27. The apparatus of claim 26, wherein the device is configured to generate a signal associated with a level of physical response at or near the body part.

28. The apparatus of claim 26, wherein the processor is configured to receive one or more signals from the one or more devices.

29. The apparatus of claim 26, further comprising an input terminal in communication with the processor, the input terminal configured to provide at least one parameter to the processor.

30. The apparatus of claim 26, further comprising a monitor in communication with the processor, the display configured to show a visual representation of the physical response.

31. The apparatus of claim 26, further comprising a signal amplifier in communication with the processor.

32. The apparatus of claim 26, wherein the at least one device comprises an electromyography (EMG) electrode or an electroencephalography (EEG) electrode.

33. The apparatus of claim 26, further comprising a delivery device configured to deliver a therapeutic agent to the body part, the delivery device in communication with the processor.

34. The apparatus of claim 23, wherein the delivery device comprises a programmable dispenser.

35. The apparatus of claim 23, wherein the delivery device comprises a catheter.

36. A computer readable medium on which is encoded computer-executable program code to diagnose a location of pain in a subject, the program code comprising:
program code for receiving a first signal via a device, the first signal associated with a first level of a physical response at or near the portion of a body part believed to be causing, or associated with, the generation of pain; and
program code for determining a location of a body part actually causing the generation of pain based at least in part on the physical response.

37. The computer-readable medium of claim 36, further comprising:
program code for receiving a control signal via a control device, the control signal associated with a physical response at or near the portion of a body part believed to be free of pain; and
program code for comparing the first signal and the control signal to provide a level of physical response caused by, or associated with, the generation of pain.

38. The computer-readable medium of claim 37, further comprising:
program code for comparing the signals received from a plurality of devices positioned at or near a portion of an internal body part believed to be causing, or associated with, the generation of pain to provide a level of physical response caused by or associated with the generation of pain.

39. The computer-readable medium of claim 36, further comprising:
program code for correlating the first level of physical response received via the device to a subject's subjective perception of pain.

40. The computer-readable medium of claim 36, further comprising:
program code for determining how a differential in the physical response the change in the signal or signals received from the first tool at a first time point as compared to the signal or signals received at a second time point corresponds to the location of the body part actually causing the generation of pain.

41. The computer-readable medium of claim 36, further comprising program code for generating a treatment signal, wherein the treatment signal is configured to cause a distribution of a therapeutic agent to the body part.
